(19) United States
(12) Patent Application Publication
(10) Pub. No.: US 2012/0088691 A1
(43) Pub. Date: Apr. 12, 2012

(54) HIGHLY MULTIPLEXED REAL-TIME PCR USING ENCODED MICROBEADS

(76) Inventors: Gao Chen, Hawthorne, CA (US); Winston Z. Ho, Hacienda Heights, CA (US)

(21) Appl. No.: 13/189,142

(22) Filed: Jul. 22, 2011

Related U.S. Application Data
(60) Provisional application No. 61/404,730, filed on Oct. 8, 2010.

(51) Int. Cl. C40B 30/10; C12M 1/40; C40B 60/12; C12Q 1/68

(52) U.S. Cl. 506/12; 435/6.12; 435/287.2; 506/39

(57) ABSTRACT

Multiple probes/primers expand the capability of single-probe real-time PCR. Multiplex real-time PCR uses multiple probe-based assays, in which each assay have a specific probe labeled with a unique fluorescent dye, resulting in different observed colors for each assay. Real-time PCR instruments can discriminate between the fluorescence generated from different dyes. Different probes/primers are labeled with different dyes that each have unique emission spectra. By combining the encoded microbeads and real-time PCR amplification, it is possible to increase the multiplexity of PCR experiments to a very large number, such as 128 with 7 digit or 4,096 with 12-digit barcode. Oligonucleotide probes/primers labeled with encoded microbeads offer the ability to monitor the reaction kinetics of each probe which is tagged with barcoded beads.

### CROSS-REFERENCE TO RELATED APPLICATIONS

This application claims benefit of U.S. Provisional application Ser. No. 61/404,730 filed Oct. 8, 2010 the disclosure of which is incorporated herein by reference.

### FIELD OF INVENTION

This invention relates to highly multiplexed real-time polymerase chain reaction (PCR) assays capable of simultaneously identifying and monitoring many targets, such as 4,096, in a sample in solution phase.

### BACKGROUND OF THE INVENTION

PCR is one of the most widely used methods in the current nucleic acid bioassays. Multiplex PCR is based on the simultaneous amplification of more than one target sequence in a single reaction. Specifically, duplex PCR is the amplification of two target sequences in one reaction, triplex PCR is the amplification of three targets, and so on. Most of multiplex PCR reactions are analyzed by end-point or post-PCR; one or more target templates are amplified in a solution phase, then the resulting amplified samples are analyzed by gel-electrophoresis or solid phase microarray.

The use of agarose gels for detection of PCR product is tedious and very time consuming. It is a qualitative method and is difficult to provide quantitative information. Solid phase microarrays or biochips are planar surface presenting with multiple spots; each spot, 100 um, is immobilized with a specific DNA probe. Up to tens of thousands spots can be spotted or printed on a glass, membrane, microplate surface. The typical surface area is approximately 1 cm×1 cm. Each spot is scanned and identified by its XY location or coordinate on a planar surface.

Microarray assaying is very powerful method for bio-discovery to screen specific biomarkers from a large number of potential targets. However, the use of microarrays is also known to have three major drawbacks: 1. Due to the large surface, the solid phase reaction kinetic is very slow; it typically takes 16 hours for the reaction to complete. The reaction is diffusion limited, which means the target molecules need to travel a very long distance in order to react with the DNA probes on specific spots. 2. Inflexibility: once the probes are spotted, it is nearly impossible to add or remove probes. If one wants to increase the number of spots from 100 to 101, one needs to re-spot the whole surface again. 3. Spotting the probes on the surface is a time consuming process and very costly. Because of these drawbacks, microarrays are rarely used in clinical diagnostic industry, where speed, flexibility, and cost are very important factors.

Real-time PCR (also known as quantitative PCR (qPCR)), in contrast to the regular PCR, offers the ability to detect amplification reaction in real time. The reaction kinetics can be monitored in the liquid phase while the amplification process still proceeding. Real-time chemistries allow for the detection of PCR amplification during the early phases of the reaction. Based on the increase of the fluorescence intensity from a specific dye, the concentration of the target can be determined even before the amplification reaches its plateau. The experiment can be completed within 30 minutes and no post PCR process is needed. Measuring the kinetics of the reaction in the early phases of PCR provides a distinct advantage over traditional PCR detection.

The use of multiple probes expand the capability of single-probe real-time PCR. Multiplex real-time PCR uses multiple probe-based assays, in which each assay has a specific probe labeled with a unique fluorescent dye, resulting in different observed colors for each assay. Real-time PCR instruments can discriminate between the fluorescence generated from different dyes. Different probes are labeled with different dyes that each have unique emission spectra. Unfortunately, due to the large fluorescence bandwidth of each fluorescence dye, the degree of multiplexing in real-time PCR is limited to four or six. Spectral signals are collected with discrete optics, passed through a series of filter sets, and collected by an array of detectors. Spectral overlap between dyes is corrected by using pure dye spectra to deconvolute the experimental data by matrix algebra.

All real-time PCR systems rely upon the detection and quantitation of fluorescent dyes or reporters, the signal of which increase in direct proportion to the amount of PCR product in a reaction. For example, in the simplest and most economical format, that reporter is the double-strand DNA-specific dye SYBR® Green (Molecular Probes). SYBR Green is a dye that binds the minor groove of double stranded DNA. When SYBR Green dye binds to a double stranded DNA, the fluorescence intensity increases. As more double stranded amplicons are produced, SYBR Green dye signal will increase.

The two most popular alternatives to SYBR Green are TaqMan® and molecular beacons (hairpin probe), both of which are hybridization probes relying on fluorescence resonance energy transfer (FRET) for quantitation. TaqMan® Probes are oligonucleotides that contain a fluorescent dye, typically on the 5′ base, and a quenching dye, typically located on the 3′ base. When irradiated, the excited fluorescent dye transfers energy to the nearby quenching dye molecule rather than fluorescing, resulting in a nonfluorescent substrate. TaqMan® probes are designed to hybridize to an internal region of a PCR product. During PCR, when the polymerase replicates a template on which a TaqMan® probe is bound, the 5′ exonuclease activity of the polymerase cleaves the probe. This separates the fluorescent and quenching dyes and FRET no longer occurs. Fluorescence increases in each cycle, proportional to the rate of probe cleavage. Molecular beacons (U.S. Pat. No. 5,118,801) also contain fluorescent and quenching dyes, but FRET only occurs when the quenching dye is directly adjacent to the fluorescent dye. Molecular beacons are designed to adopt a hairpin structure while free in solution, bringing the fluorescent dye and quencher in close proximity. When a molecular beacon hybridizes to a target, the fluorescent dye and quencher is separated, FRET does not occur, and the fluorescent dye emits light upon irradiation. Unlike TaqMan® probes, molecular beacons are designed to remain intact during the amplification reaction, and must rebind to target in every cycle for signal measurement. Multiplex PCR allow multiple DNA species to be measured in the same sample, since fluorescent dyes with different emission spectra may be attached to the different probes. Multiplex PCR allows internal controls to be co-amplified and permits allele discrimination in single-tube, homogeneous assays. For real time monitoring, the amplification reactions are performed by means of rapid thermocycling, wherein one cycle is less than one minute. The instrumental basis for such a rapid thermocycling is provided e.g. by the LightCycler (Roche Molecular Biochemicals) and disclosed in WO 97/46707 and WO 97/46712.

U.S. Pat. No. 7,118,867 disclosed a method for quantitative multiplex PCR of two targets whereby one target is present in at least 100-fold molar excess over that of the other target. U.S. Pat. No. 7,183,056 disclosed an invention directed to an improved multiplex PCR method for obtaining at least two PCR products from one PCR solution. In the multiplex PCR method for having at least two DNA amplified products from a sample positioned in a PCR equipment, the object of the present invention is to provide a novel multiplex PCR method characterized in that a primer annealing temperature and an extension time be changed per cycle with constant periods.

Multiplex fluorescent tags are used in conjunction with qPCR system, including FAM, TET, ALEXA 532, ALEXA 647, TAM, ROX, SYBR Green, Cy3, Texas Red and others. But all fluorescence spectra from these dyes are very broad (40 nm-60 nm of spectral bandwidth) and the number of multiplicity is very limited due to spectral overlap. That is the reason that the number of multiplexing in real-time PCR is limited to four or six. When the number of multiplexing is over six, the current qPCR technology presents an extreme challenge. While tremendous interest for multiplexing PCR, the fundamental limitation is the number of fluorescence dyes which can be used simultaneously. As the number of fluorophore increases, the broad fluorescence spectra start to overlap, and the optics and detection system become more complicate. It commonly uses one detector for one specific fluorophore. Six fluorophores will require six filter sets and six detectors which significantly increases the cost of the system.

Of interest to the present application are developments made in microbead technology. Micro bead technology potentially overcomes many of the problems of microarray technology and provides flexibility of library content and amount of beads or bead type in an analysis. Due to its small volume (in the range of picoliter per bead), thousands of beads can be incubated with a very small amount of sample. A number of encoding strategies have been demonstrated include particles with spectrally distinguishable fluorophore, fluorescent semiconductor quantum dots, and metallic rods with either bar coded color (absorption) stripes or black and white strips. Both fluorescence and barcode color strip beads are identified by optical detection in reflective or emissive configuration. The difficulties of reflection configuration are (1) the optical reflection yield is low, especially when the beads are in micrometer scale, (2) the light collection efficiency is poor, and (3) for fluorescence-based encoded beads, the fluorescence bands are very broad and overlapped, thus limit the potential code number. Another drawback of fluorescence-based bead is that most bead-based assay rely on fluorescence readout, thus creating more fluorescence spectral or intensity interference. In the case of multi-metal (Au, Pt, Ni, Ag, etc) color micro rods, the encoding scheme suffers from the difficulty of manufacturing and the number of colors, based on different metal materials, is limited.

U.S. Pat. No. 6,773,886 issued on Aug. 10, 2004 discloses a form of bar coding comprising 30-300 nm diameters by 400-4000 nm multilayer multi metal rods. These rods are constructed by electrodeposition into an alumina mold; thereafter the alumina is removed leaving these small multilayer objects behind. The system can have up to 12 zones encoded, in up to 7 different metals, where the metals have different reflectivity and thus appear lighter or darker in an optical microscope depending on the metal type whereas assay readout is by fluorescence from the target, and the identity of the probe is from the light dark pattern of the barcodes.

U.S. Pat. No. 6,630,307 issued on Oct. 7, 2003 discloses semiconductor nano-crystals acting as a barcode, wherein each semiconductor nano-crystal produces a distinct emissions spectrum. These characteristic emissions can be observed as colors, if in the visible region of the spectrum, or may be decoded to provide information about the particular wavelength at which the discrete transition is observed.

U.S. Pat. No. 6,734,420 issued on May 11, 2004 discloses an identification system comprising a plurality of identifiable elements associated with labels, the labels including markers for generating wavelength/intensity spectra in response to excitation energy, and an analyzer for identifying the elements from the wavelength/intensity spectra of the associated labels.

U.S. Pat. No. 6,350,620 issued on Feb. 26, 2002, discloses a method of producing a micro carrier by placing a bead between a nickel plate on which the barcode has been electroformed and a second plate, and compressing the barcode onto the surface of the bead to form a microcake-like particle with a barcode.

U.S. Pub. No. US2005/0003556 A1 discloses an identification system using optical graphics, for example, bar codes or dot matrix bar codes and color signals based on color information signal for producing the affinity reaction probe beads. The color pattern is decoded in optical reflection mode.

U.S. Pub. No. US2005/0244955 discloses a micro-pallet which includes a small flat surface designed for single adherent cells to plate, a cell plating region designed to protect the cells, and shaping designed to enable or improve flow-through operation. The micro-pallet is preferably patterned in a readily identifiable manner and sized to accommodate a single cell to which it is comparable in size.

Magnetic beads are used widely in high throughput automated operation. The magnetic beads, are paramagnetic, that is, they have magnetic property when placed within a magnetic field, but retain no residual magnetism when removed from the magnetic field. This allows magnetic collection of microbeads and resuspension of the beads when the magnetic field is removed. Collection and resuspension of the digital magnetic beads can be repeated easily and rapidly any number of times. The common robotic automation is simply putting a 96-well, 384-well or 1536-well microplate on a magnetic stand facilitated with magnetic pins to activate the magnetic field. This enables washing of unbound molecules from the beads, changing buffer solution, or removing any contaminant in the solution. For example, in the case of DNA or RNA assays, the unbound or non-specific nucleotides can be removed after hybridization. While in the case of protein assay, the unbound or non-specific antibodies or antigens can be removed after the antibody-antigen reaction. Extensive washing often required during molecular biology applications to be conducted swiftly, efficiently, and with minimal difficulty. While magnetic beads are widely used in the bioassays, no magnetic beads with high density barcode are available.

Applicant's prior PCT Patent Application No. PCT/US08/08529 discloses a digitally encoded magnetic micro bead that provides high optical contrast and high signal-to-noise for reliable decoding, and also provides magnetic property for high-throughput automated washing in the microplate format. The application also discloses a decoding method including a data process that is relatively simple, robust, rapid and accurate.

There are microsphere beads which incorporated with different fluorescence dyes (Luminex Corporation) or quantum dots (Life Technologies) for multiplex assays. These methods are based on the ratio of the fluorescence intensities from different dyes to provide multiple identification codes. The fluorescence signals are excited with a focus illumination beam on a microsphere. Microsphere beads are detected by a flow cytometer. Microbeads are withdrawn from the microplate, passed through a small microfluidic channel and fluorescently detected one by one in a high speed flow system. Thousands of microbeads passed through an optical detection zone, then go into a waste system. Although these microbeads offer multiplexed codes, the flow cytometer system is not comparable with PCR system. Flow cytometer facilitated with tubing, pump, circulation system, thus it is not suited for repeated thermocycling experiments. In the PCR system, the microbeads need to be cycled at three define temperatures; and the sample is confined in a highly temperature regulated microwell or microtube.

### SUMMARY OF THE INVENTION

The invention provides a multiplex PCR and amplification assays capable of screening or detecting a large number of targets in a sample in real time. Although the focus of this invention is real-time detection, the methods of the invention can also be used for end-point or post PCR detection.

The present invention relates to real-time PCR multiplexing directed to the use of encoded microbeads including, but not limited to, barcoded magnetic beads (BMB). Encoded microbeads provide an open-ended digital multiplex platform, are encoded with a high contrast pattern on micro particles, which simplify multiplexed immuno- and molecular diagnostic assays while offering high throughput, high accuracy, and cost savings. Depending on the number of digits (N) on a single microbead, the number of unique identification codes, 2N, can be easily extended from 32 (=25) to 4,096 (=212). When each probe or primer is tagged with a specific barcode bead, an unlimited or open-end number of probes can be used for monitoring unlimited number of target in a sample. Because of the size of labeled microbeads, thousands of labeled microbeads are in liquid suspension during reaction. Preferably the microbeads have lengths ranging from 50 μm to 300 μm, widths ranging from 10 μm to 100 μm and thicknesses from 2 μm to 50 μm although the beads can be larger or smaller as might be desired for a particular application. The reaction kinetics of labeled microbeads in liquid phase is much faster than that in solid phase microarray. According to one aspect of the invention, the total reaction time can be reduced from 10 hours to less than 1 hour. Utilizing labeled microbeads, only one fluorescence dye or reporter is needed. Fluorescence is used for reaction monitoring, while barcode is used for probe identification. Every single bead which carries a specific barcode is optically decoded, and fluorescence is detected after each thermocycle. Labeled microbeads offer the multiplex flexibility by simply adding whatever new beads with specific probes into the cocktail or reaction mix. The ability to multiplex PCR by probe tagged with encoded microbeads expands the power of real-time amplification analysis. The potential applications are viral quantitation, gene expression, drug therapy efficacy, biomarker validation, DNA damage measurement, quality control and assay validation, pathogen detection, genotyping, and others.

Encoded microbeads and apparatus and systems for their analysis are disclosed herein but reference is hereby made to the following U.S. patents and pending U.S. patent applications the disclosure of which are hereby incorporated by reference including the disclosure of U.S. patent application Ser. No. 12/069,720, filed Feb. 11, 2008, and those of U.S. Pat. No. 7,871,770, and U.S. Pat. No. 7,858,307. Also incorporated herein are the disclosures of U.S. patent application Ser. No. 12/386,369 filed Apr. 17, 2009, U.S. patent application Ser. No. 12/832,972 filed Jul. 8, 2010 and U.S. patent application Ser. No. 12/576,076 filed Oct. 8, 2009.

Presented herein is a multiplex real-time liquid phase PCR amplification assay capable of monitoring PCR kinetics and quantifying the concentration of 1 to 1,024 targets or microbial organisms in a sample in a microplate, however only one fluorophore emission signal is needed.

This invention provides 1 to 4,096 real-time liquid phase labeled microbead—PCR amplification point tests and capable of monitoring PCR kinetics and quantifying the concentration of targets after each PCR cycle, however only one fluorophore emission signal is needed.

This invention provides a multiplex real-time liquid phase labeled microbead PCR amplification assay based on encoded microbeads capable of monitoring PCR kinetics and quantifying the concentration of 1 to 4,096 targets or microbial organism in a sample, however only one fluorophore emission signal is needed. The labeled microbead is very stable in a wide temperature range (between −20° C. to 98° C.), which makes it very suitable for PCR reactions. The labeled microbead PCR cycle involved the steps of denaturing, annealing, extension, and optical imaging of labeled microbeads on a flat surface. Here the optical imaging comprising of barcode imaging and fluorescence imaging.

According to one aspect of the labeled microbead tagged PCR probes are used in the practice of a PCR method comprising the following four steps: denaturation, annealing and extension, and an additional optical imaging step. First, the genetic material is denatured, converting the double stranded DNA molecules to single strands. The primers are then annealed to the complementary regions of the single stranded molecules. In the third step, they are extended by the action of the DNA polymerase. All these steps are temperature sensitive and the common choice of temperatures is 94° C., 60° C. and 70° C. respectively. In the fourth step, optical detection is performed after the annealing or extension step. The labeled microbeads will be settled down to the bottom of the plate for optical imaging. A typical thermocycle requires only 30 to 60 seconds; an amplification reaction with 30 cycles is usually complete in about 30 minutes. The liquid phase labeled microbeads PCR is performed inside a microplate.

This invention further provides a multiplex PCR or amplification assay capable of screening or detecting a large number of targets or microbial organism in a sample in real time. Each specific probe/primer is labeled or tagged with an encoded bead, such as a barcoded bead, which can be decoded optically. As used herein reference to an oligonucleotide “probe” and “primer” can be synonymous depending upon the application to which it is applied with probe indicating an oligonucleotide presenting a specific sequence capable of hybridizing under selected conditions to a complementary sequence and a primer indicating such an oligonucleotide capable of hybridizing to a complementary sequence which is further intended to be subjected to chain extension in the presence of appropriate ingredients such as polymerases, nucleotides (dNTPs) and buffers Only one fluorescence reporter is needed and tens, hundreds, or thousands of amplification reactions can be measured simultaneously in a sample. By decoding the barcode pattern on the bead, one can determine which probe is immobilized on the bead. By detecting the fluorescence intensity on the bead, one can monitor the amplification kinetics of each target after each reaction cycle. The amplification kinetics: fluorescence intensity versus number of cycle, for each encoded microbead is reported. Because both barcode image and fluorescence image can be processed rapidly, highly multiplex amplification reactions can be monitored simultaneously in real-time for a sample.

The invention provides a highly multiplex real-time amplification method comprising: (a) a plurality of encoded microbeads, molecular probes or primers, fluorescence reporter, PCR mix, and target nucleic acids; (b) wherein each specific molecular probe or primer is immobilized on the encoded microbeads with a specific identity; (c) the encoded microbeads produce fluorescence signal, which is generated from the amplification reactions of the molecular probe or primer, the fluorescence reporter, the PCR mix, and the target nucleic acids, if present in the sample; and (d) the encoded microbeads are decoded and the fluorescence signals from the encoded microbeads are monitored by optical imaging on an optically clear flat surface after each heating and cooling cycle.

This invention provides a labeled encoded microbead highly multiplex real-time amplification analyzer comprising: a thermocycler, a bead mixing device, a microwell, and reagent kits; the reagent kit, in the microwell, comprising a plurality of encoded microbeads, molecular probes or primers, fluorescence reporter, PCR mix, and target nucleic acids. Each specific molecular probe or primer is immobilized on the encoded microbeads with a specific barcode number. The encoded microbeads produce fluorescence signal, which is generated from the amplification reactions of the molecular probe or primer, the fluorescence reporter, the PCR mix, and the target nucleic acids, if present in the sample, The thermocyler has the ability to repeatedly heating and cooling the reagent kits in the microwell; and the microwell is optical clear and has a flat bottom. The bead mixing device can be a plate shaker which can be turned on to suspend the encoded microbeads in solution or turned off to settle the encoded microbeads on the bottom of the microwell. According to one embodiment of the invention, the encoded microbeads are magnetic microbeads which can be alternatively suspended or settled to the bottom of the microwell by the application of a magnetic field. After each heating and cooling cycle, when beads are settled on the bottom of the microwell, the encoded microbeads are decoded and the fluorescence signals are monitored by optical imaging.

According to one embodiment, the invention provides a method for real-time polymerase chain reaction (PCR) detecting of multiple target molecules in a solution, the method comprising: (a) preparing of a plurality of samples, each containing multiple labeled microbeads, a fluorescence probe and target molecules; each encoded microbead having a specific encoding pattern such as a barcode pattern; (b) placing each sample in one of a plurality of sample wells of a thermal cycler instrument, each sample well having a flat surface; stimulating a reaction using the thermal cycle instrument; (c) taking an optical image and a fluorescence image of the labeled microbeads when the barcode magnetic beads settling down to the flat surface of the sample wells, the thermal cycler in operating; (d) decoding the specific barcode pattern and measuring the fluorescence intensity of each encoded microbead with the barcode optical image and the fluorescence image, respectively; and (e) quantifying specific target molecules based on the fluorescence intensity on the specific labeled microbeads reacting with fluorescence probe and target molecules.

The encoded microbeads of the present method for real-time polymerase chain reaction (PCR) detecting of multiple target molecules in a solution are immobilized with specific primers or probes with a specific sequence of nucleic acid sequence. According to one embodiment, the method comprises measuring the fluorescence intensity of each encoded microbead after each thermal cycling reaction or at the end of the thermal cycling reactions.

The labeled microbeads of the present method for real-time polymerase chain reaction (PCR) can have from 1 to 4,096 different barcode patterns.

The labeled microbeads of the present method for real-time polymerase chain reaction (PCR) detecting of multiple target molecules in a solution are preferably chemically and physically stable up to 98° C.

The labeled microbeads of the present method for real-time polymerase chain reaction (PCR) detecting of multiple target molecules in a solution are smaller than 1 mm in length. Preferred lengths range from 50 μm to 300 μm, with preferred widths ranging from 10 μm to 100 μm and thicknesses from 2 μm to 50 μm although the beads can be larger or smaller

According to one aspect of the invention, the bright field imaging and fluorescence image are illuminated with a light source having a wavelength between 400-750 nm. In one embodiment, the method further comprises measuring the fluorescence intensity during the annealing or extension reaction.

According to one preferred aspect of the invention, the encoded microbeads useful for practice of the method for real-time polymerase chain reaction (PCR) comprise a body having an intermediate layer sandwiched between two layers of a photoresist photopolymer material wherein the intermediate layer is coated or imbedded with a paramagnetic material and comprises an encoded pattern which is partially substantially transmissive and partially substantially opaque to light. The pattern provides a code corresponding to the micro bead, wherein the outermost surface of the micro bead comprises the photoresist photopolymer and the photoresist photopolymer is functionalized with a target or capture molecule selected from the group consist of proteins and small molecules but preferably nucleic acids that can be used in a hybridization or amplification procedure.

In one embodiment, the intermediate layer comprises a series of alternating substantially light transmissive sections and substantially light opaque sections defining the encoded pattern; wherein the light transmissive sections are defined by slits through the intermediate layer of the body, and the light opaque sections are defined by a light reflective material and/or a light absorptive material; wherein the slits comprises slits of a first width and slits of a second width, and wherein the first width represents a “0” and the second width representing a “1” in a binary code.

According to one aspect of the invention, a real-time polymerase chain reaction (PCR) detecting system for analyzing multiple target molecules in a thermal cycler, the apparatus comprising: (a) a plurality of samples, each containing multiple encoded microbeads, a fluorescence probe adapted to target molecules, each encoded microbead having a specific barcode pattern; (b) each sample in a respective one of a plurality of sample wells, each sample well having a flat surface; (c) one or two light beams to illuminate and take a barcode optical image and a fluorescence image when the encoded microbeads settling down to the flat surface of the sample wells; and (d) image software to decode the specific barcode pattern of each encoded microbead and measure the fluorescence intensity of each encoded microbead during PCR reaction. The labeled microbeads of the present real-time PCR detecting apparatus are immobilized with specific primer or probe with specific sequence of nucleic acid sequence. In one embodiment, the real-time PCR detecting apparatus further comprises measuring the fluorescence intensity of each encoded microbeads after each thermal cycling reaction or at the end of thermal cycling reactions.

To enhance the surface chemistry, microbeads can be mixed in solution by various methods, such as rotation, shaking, acoustic wave, magnetic field, and ultrasound mixing. Electromagnetic devices are used for magnetic beads mixing in the automatic robotic systems. Acoustic wave and ultrasound can be used for liquid mixing. Sample plate or sample microwell should be sealed with an optically clear sealing film before put into themocycler. Heating and cooling can be achieved through various rapid heating/cooling sources. Peltier pump and can be used in the PCR system to control the heating cycle. Sample plates or tubes are inserted into the heating block controlled by the Peltier pump. Hot and cold airflow are also used to heat or cool the sample chamber.

Specifically, the invention provides an amplification method for the analysis of nucleic acid samples comprising the steps of: contacting said sample with a nucleic acid primer or probe capable of hybridizing with a selected target nucleic acid under chain extension conditions wherein said primer or probe is linked to a microbead presenting an optically detectable code specific for said probe; conducting polymerase mediated chain extension such that the presence of target nucleic acid results in the presence or absence of a detectable signal physically associated with the encoded microbead; settling said encoded microbeads to the bottom of a well; and measuring the signal associated with said encoded microbead at more than one point in time. Note that as used herein nucleic acid “primer” and “probe” can generally be used interchangeably and are usually defined by their context. In generally a “probe” is an oligonucleic acid sequence which is used to hybridize to a target while a “primer” hybridizes to a target and is then subject to chain extension in the presence of polymerase and single nucleic acids. According to various aspects of the invention the encoded beads are encoded with a bar-code and have 2 to 4,096 different barcode patterns; are chemically and physically stable up to 98° C. and are smaller than 1 mm in length.

The invention also provides a system for carrying out real-time polymerase chain reaction (PCR) detecting for analyzing multiple target molecules in a thermal cycler, the system comprising: a plurality of samples, each sample containing multiple encoded microbeads, a fluorescence probe adapted to target molecules, each encoded microbead having a specific barcode pattern; each sample in a respective one of a plurality of sample wells, each sample well having a flat surface; one or two light beams to illuminate and take a barcode optical image and a fluorescence image when said encoded microbeads settle down to the flat surface of said sample wells; and an image software to decode the specific barcode pattern of each encoded microbead and measure the fluorescence intensity of each encoded microbead during a PCR reaction. According to one preferred aspect of practice bright field imaging and the fluorescence image are illuminated with a light source having a wavelength between 400-750 nm.

According to another aspect of the invention the beads are magnetic beads.

According to a further aspect of the invention the microbeads comprise: a body comprising an intermediate layer sandwiched between two layers of a photoresist photopolymer material wherein the intermediate layer is coated or imbedded with a paramagnetic material and comprises an encoded pattern which is partially substantially transmissive and partially substantially opaque to light, wherein said pattern provides a code corresponding to the micro bead, wherein the outermost surface of the micro bead comprises said photoresist photopolymer and said photoresist photopolymer is functionalized with a target or captures a molecule selected from the group consisting of proteins, nucleic acids and small molecules. In one preferred embodiment the intermediate layer comprises a series of alternating substantially light transmissive sections and substantially light opaque sections defining the encoded pattern, wherein the light transmissive sections are defined by slits through the intermediate layer of the body, and the light opaque sections are defined by a light reflective material or a light absorptive material; wherein the slits comprises slits of a first width and slits of a second width, and wherein the first width represents a “0” and the second width representing a “1” in a binary code.

A magnetic field is used to settle said encoded microbeads to the bottom of a well and/or to re-suspend the beads after they have been settled.

Various amplification methods may be used in practice of the invention including, but not limited to those involving multiple cycles of cycles of thermally denaturing and annealing double stranded nucleic acids such as polymerase chain reaction (PCR). Real time PCR assays can then be carried by measuring the signal associated with said encoded microbeads is measured at the same point (preferably at the conclusion) of two or more thermal cycles.

Other amplification methods may be used including those which do not depend upon thermal cycling such as helicase dependent amplification (HDA) in which a sample is contacted with helicase and the target nucleic acid is amplified by helicase dependent amplification. Such an amplification method does not have thermal cycles but the signal associated with the encoded microbeads can measured at the multiple selected points in time to provide a curve indicative of the quantity of target present in a sample.

The invention also provides methods of performing an isothermal cleavase amplification by which an analysis of nucleic acid samples is carried out comprising the steps of: contacting said sample with oligonucleotides capable of forming an invasive cleavage structure in the presence of said target sequence and an agent for detecting the presence of an invasive cleavage structure; and exposing said sample to said oligonucleotides and said agent under conditions such that said invasive cleavage structure is cleaved by said agent and detecting the cleavage product by the hybridization of such cleavage product to a second probe capable of hybridizing to said cleavage product which second probe is linked to a microbead presenting an optically detectable code specific for said second probe and whereby the presence or absence of hybridization of said cleavage product of said invasive cleavage structure to said second probe is indicated by a detectable signal, settling said encoded microbeads to the bottom of a well; and measuring the signal associated with said encoded microbead. The second probe can be a fluorescence resonance energy transfer (FRET) probe and the signal associated with said encoded microbeads is measured at the multiple selected points in time.

### BRIEF DESCRIPTION OF THE DRAWINGS

For a fuller understanding of the scope and nature of the invention, as well as the preferred mode of use, reference should be made to the following detailed description read in conjunction with the accompanying drawings. In the following drawings, like reference numerals designate like or similar parts throughout the drawings.

FIG. 1 is a schematic illustration of the physical configuration of the instrument system;

FIG. 2(a) is a schematic flow diagram of the image process undertaken by the system instrumentation for bead decoding and fluorescence detection, including overlapping and stitching image frames;

FIG. 2(b) is a flow chart illustrating an example method 200 of imaging and decoding a bead;

FIG. 2(c) illustrates an exemplary flowchart of a segmentation based algorithm 250;

FIG. 2(d) is an exemplary flowchart 260 illustrating an enhancement routine;

FIG. 2(e) is an exemplary flowchart 280 illustrating a possible grayscale processing routine;

FIG. 2(f) is an exemplary flowchart 300 illustrating a routine for performing segmentation and area filter of bars;

FIG. 2(g) is an exemplary flowchart 320 illustrating a routine for performing dilation and area filtering of beads;

FIG. 2(h) is an exemplary flowchart 340 illustrating a routine for performing a pattern search and decoding of barcodes;

FIG. 3 shows the transmitted digital signal of a barcoded bead representing 0011111001 on an image camera;

FIG. 4 (a) illustrates overlapping image frames for one well; (b) is a schematic diagram explaining overlapping image frames to account for partial beads at boundaries; (c) illustrates overlapping image frame data sent to processing module;

FIG. 5 shows the segmentation based algorithm comprises of five main sub-processes (1) Enhancement of bright field image (2) Grayscale threshold (3) Segmentation and area filtering of bars (4) Dilation and area filtering of beads, and (5) Pattern search and decoding of barcodes;

FIG. 6(a) is a diagram illustrating barcode decoding using a grid search routine in accordance with one embodiment of the present invention;

FIG. 6(b) is a diagram illustrating bead image determination using a grid search routine in accordance with one embodiment of the present invention;

FIG. 6(c) is a diagram illustrating bar image determination using a grid search routine in accordance with one embodiment of the present invention;

FIG. 7 is an illustration of fluorescence calculation of the beads using the area between the two concentric rectangles in dotted lines;

FIG. 8 is an exemplary image of decoded beads in a single image frame showing the bead numbers, barcode of the beads and fluorescence value of the beads;

FIG. 9 is an illustration of background correction and thresholding;

FIG. 10 shows area filtering for bars to detects objects equal to the area of larger bars;

FIG. 11 shows filtering with bounding box in order to remove stray objects at the edges of the beads;

FIG. 12 shows dilation of bars in order to detect beads;

FIG. 13 shows area filtering of beads in order to remove overlapping or partial beads;

FIG. 14 shows geometric matching for barcode detection using computer generated template barcodes;

FIG. 15 illustrates the shortened bead with no spaces between code segment;

FIG. 16 illustrates an example computing device that may be used in an image decoding system;

FIG. 17 illustrates the reactions for preparing probe or primer coupling to encoded microbeads. Each probe or primer can be coupled to a barcoded magnetic bead (BMB) with a specific barcode.

FIG. 18 illustrates one cycle of BMB-based multiplex PCR/or real-time PCR with double-stranded DNA-binding dyes as reporters. (a) for target N, forward primer N is coupled to one barcoded BMBs N. (b) for target N, forward and reverse primer pair N are coupled to one barcoded BMBs N.

FIG. 19 illustrates the process for BMB-based multiplex qPCR/RT-PCR using hairpin primers labeled with fluorophore.

FIG. 20 illustrates the process for BMB-based multiplex qPCR/RT-PCR using molecular beacons probes.

FIG. 21 illustrates the images of the BMBs at the bottom of the flat bottom microplate.

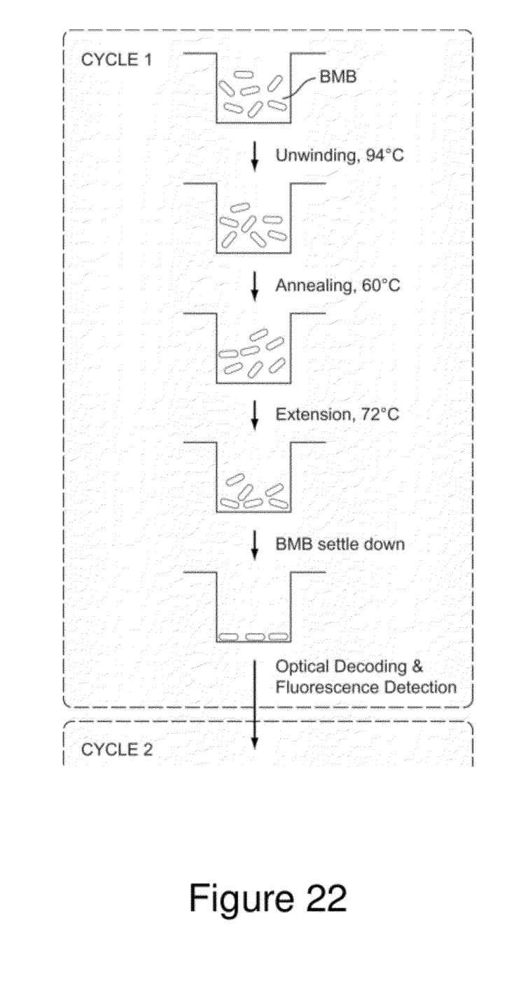

FIG. 22 illustrates the typical PCR steps with BMB tagged probes. The biochemistries occurred on the surface of BMB.

FIG. 23 (a) illustrates the heating and cooling elements of the BMB-based multiplex real-time PCR detection system, and (b) illustrates the alternative heating and cooling elements of the BMB-based multiplex real-time PCR detection system,

FIG. 24 illustrates the fluorescence signal increase as function of the amplification cycle and detected on the bottom of the microplate.

FIG. 25 illustrates the optical components of the real-time analytical system.

FIG. 26 illustrates the fluorescence signal as function of the number of cycle for different BMB tagged with different probes.

FIG. 27 illustrates the fluorescence data as function of the number of cycle for different BMB tagged with different probes.

### DETAILED DESCRIPTION OF THE INVENTION

### Barcode Bead Optical Detection System

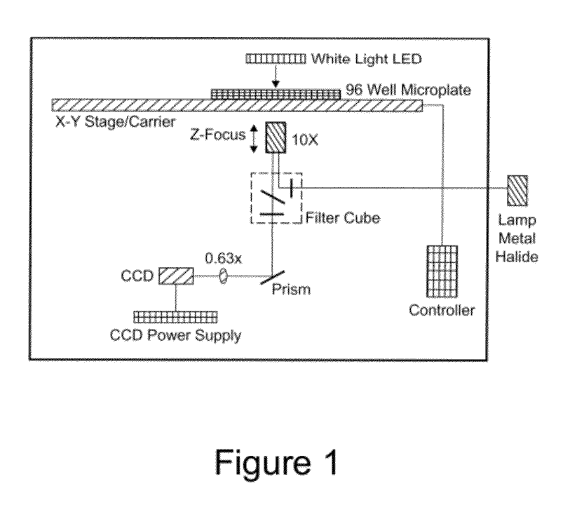

Both bar-code image and fluorescence image can be constructed on a conventional microscope or an inverted fluorescence microscope. One embodiment illustrated in FIG. 1, the digital magnetic LITAB analytical system has a white light LED source for bead pattern illumination and an optical CCD for capturing images of beads at the bottom of the support (e.g., a microwell in the illustrated embodiment). The bottom of the microwell is transparent or translucent, allowing sufficient light to pass through to the beads. A bright field light source (e.g., a white light LED) is incident from the top of the 96-well plate (e.g., via a diffuser plate to provide more uniform illumination). All current LEDs have speckle patterns or non-uniform light pattern. which cause the non-uniform light distribution and illumination. The situation becomes worse when the light is illuminated near the edge or the wall of the microwell. A light diffuser film has been made and implemented on top of the plate as a plate scaler or attached on the microplate cover. The diffuser film homogenizes the LED light pattern, thus every image frame has uniform background, which leads to much improved decoding accuracy.

A scanning or translation mechanism moves the microwell relative to the optical detector and light source to image the desired wells. The optical detector can be used for both barcode image and fluorescence detection. A 1 M pixels CCD should have sufficient pixels to resolve the barcode pattern on beads. Barcode illumination light source can be a white light, while fluorescence excitation light source need a wavelength that matches with the absorption of the fluorophore. Lens and optical filters are used to collect and select the excitation and fluorescence wavelength. By measuring the fluorescence intensity, one can identify which beads have positive biochemical reaction. By decoding the digital barcode image, one can identify which biological probe is immobilized on the surface of that microbead. The choice of light source depends on the fluorophore. For example, a Mercury light source or metal halide lamp facilitated with an optical filter cube offers UV to visible light excitation. A red diode laser (665 nm), and compact Argon Laser (488 nm) or green laser (530 nm) are common laser light sources for variety of fluorophores (e.g. phycoerythrin (PE), Cy3, and Cy 5, etc.). Optical filter sets are designed to select particular excitation and fluorescence wavelength for various fluorophores.

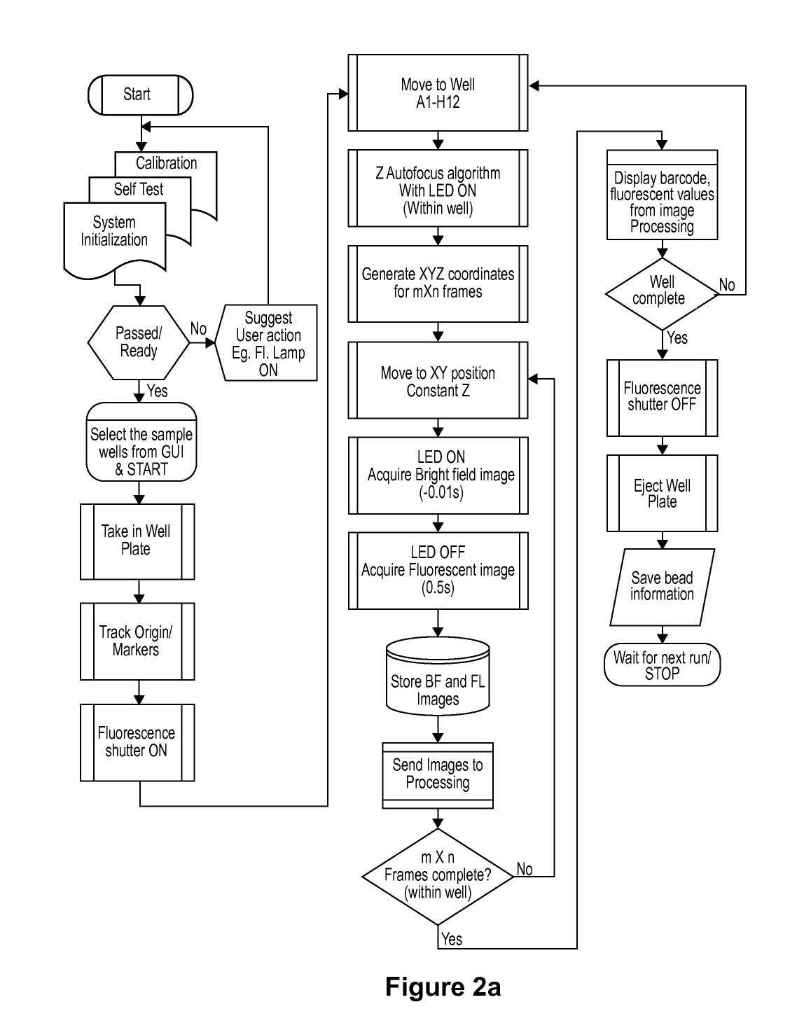

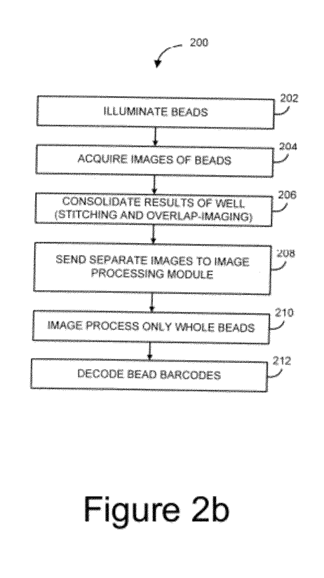

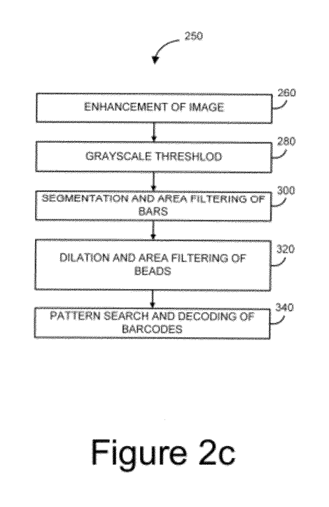

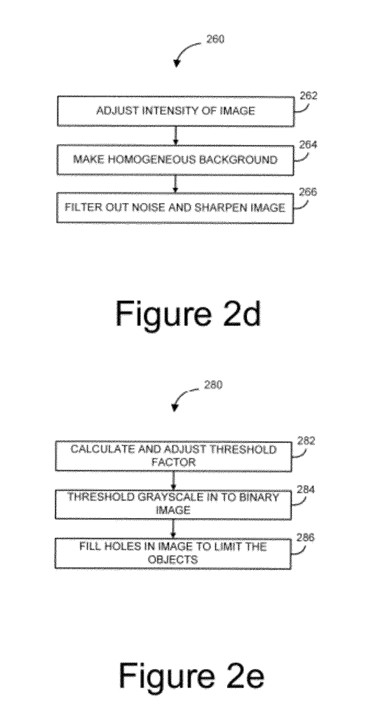

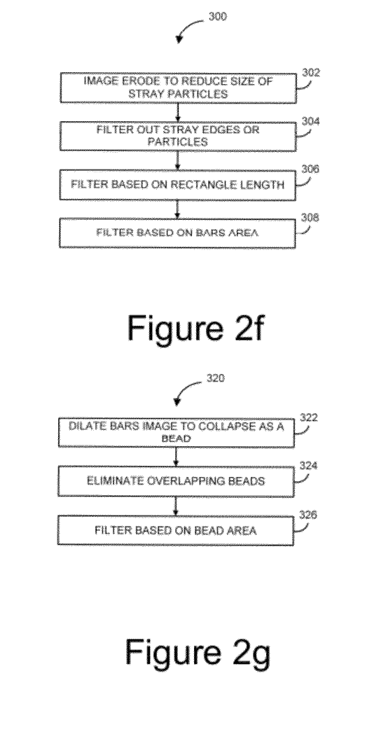

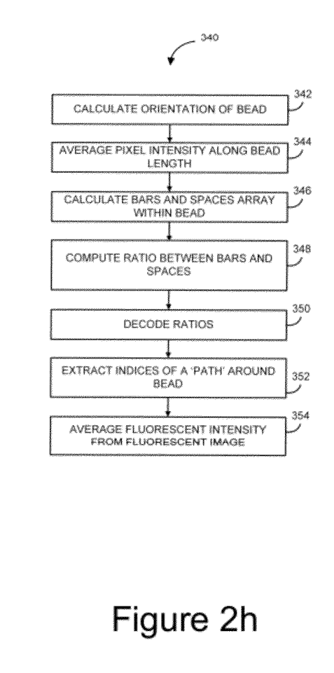

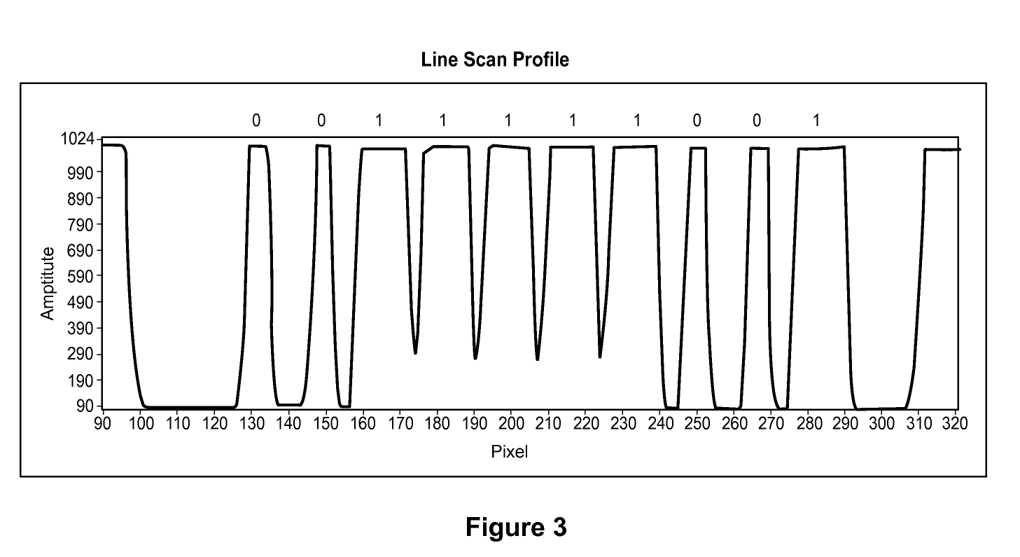

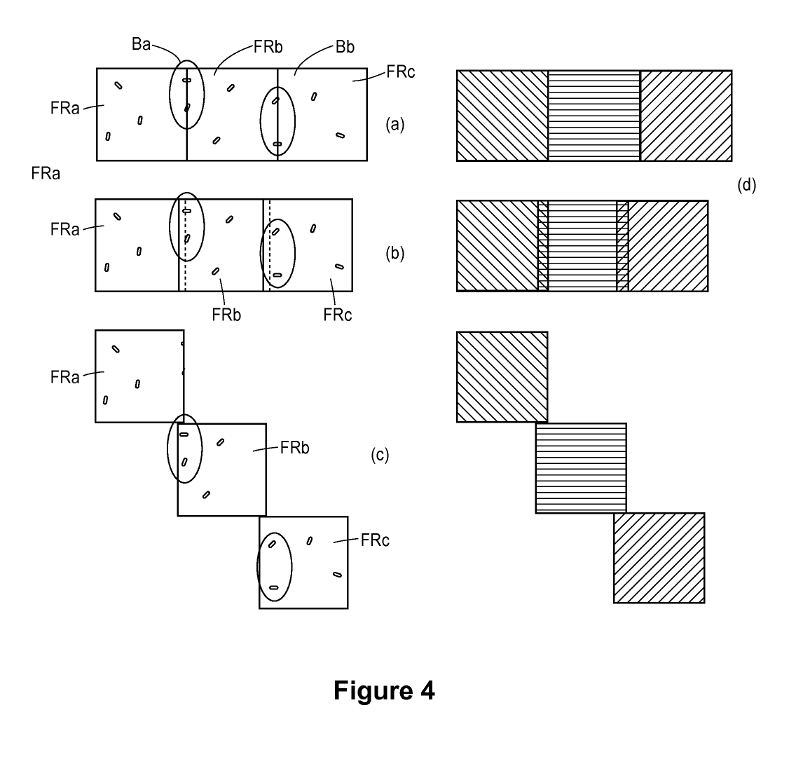

Referring to the flow diagram in FIG. 2(a) (and the system illustrated in FIG. 1), the entire image decoding and fluorescence detection system in accordance with one embodiment of the present invention is described. Upon system startup, the system goes through a startup procedure including calibration, self-test and system initialization. If startup procedure is not carried out completely or successfully, additional user action may be required to validate startup (e.g., turning fluorescence lamp on). Once startup procedure has been successfully completed, the user can initiate test procedure via a graphical user interface at the system controller/computer (e.g., select the sample wells to be imaged and processed). The sample well plate is received into the system (e.g., supported on the X-Y stage). The fluorescence shutter is turned on until all heads in the wells are imaged. If a fast shutter is used, then it does not need to be kept ON for long, which will avoid interfering with bright field image.

The X-Y stage can be controlled by the system controller to access each well (A1-H12) in sequence or as desired. At each well location, the Z-focus of the lens below the well is adjusted automatically for the CCD, with the white light LED on. Depending on the optics and focus, the number of mXn frames and the XYZ coordinates of each frame are generated. The first well is accessed based the designated X-Y position. The bright field image BF is acquired (e.g., approximately 0.01 s) with the white LED on. This BF image is stored for bead decoding offline (or can be real time if the processing system is fast enough). The white LED is then turned off, to allow acquisition of fluorescence (e.g., approximately 0.05 s). The fluorescence image FL is also stored for further image processing. The bright field BF and fluorescence FL images can be immediately sent for image processing by the decoding and fluorescence analysis processes to identify the beads and determine fluorescence values as each frame is imaged

The randomly oriented microbeads can be decoded on a support, such as a slide or in the bottom of a microplate by imaging processing method. When beads are finally settled down and distributed on the bottom of a planar surface in a microplate, multiple beads can be decoded simultaneously with a wide viewing or scanning image camera. The microplate is a standard format for high throughput clinical assays. Each well is used for one sample and each plate holds 96, 384, or 1,536 patient samples for 96-well, 384-well, and 1,536-well, respectively. Therefore, an experiment can be performed in the microplate without taking the beads out, the image of the microbeads can be taken in the steady state with a better accuracy and sensitivity for decoding. The accuracy of decoding is very important for clinical diagnostics, because any false identification can lead to mis-diagnosis and mis-therapy. Because of the small bead size, hundreds or even thousands of beads can be displayed in the bottom of a microwell with minimal overlap. To minimize bead overlap, depend on the area of the microwell, the total number of beads is limited to a certain number. Furthermore. the non-spherical beads are tended to overlap spatially. Therefore a detection buffer solution was developed to minimize the bead overlap depending on and aggregation.

The detection buffer is composed of: (a) a bulky polymer to promote steric stabilization; (b) a compatibilizer for the copolymer; (c) salts having a solubilization effect on the biomolecules and (d) surfactant to reduce surface tension or change the surface properties (hydrophilic/hydro-phobic, interfacial tension, or charge character) of beads. The bulky polymer is chosen from natural polysaccharides. or synthetic polymers or copolymers while the compatibilizer may be chosen from copolymers containing N-vinyl pyrrolidonc (or 1-yinyl-2-pyrrolidone). Suitable surfactants may be chosen from silicone surfactants, fluoro-surfactants. anionic surfactants, cationic surfactants. or nonionic surfactants or the combination thereof.

### Acquisition of Overlapped Bright Field and Fluorescent Images

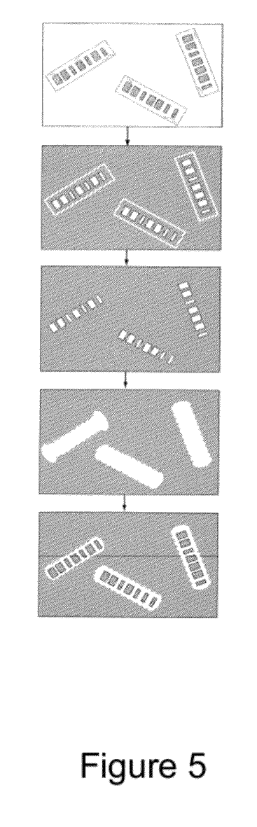

FIG. 2(b) is a flow chart illustrating an example method 200 of imaging and decoding a microbead. When the bead is illuminated (block 202) with a light beam, based on the either the “total intensity” of the transmission peak or the “bandwidth” of the transmission peak from the slit, the digital barcode either 0 or 1 can be determined by an imaging camera and a digital signal processor. As shown in the FIG. 3, the barcode patterns can be easily identified based on the peak widths. Specifically as illustrated in the embodiment shown in FIG. 3, the beads show 10-bit barcodes representing 0011111001. To image all the beads on the entire support of a single well bottom, the well bottom is scanned using the X-Y stage, to index various image frames over the entire planar area of the well bottom where beads are found (block 204). Different image frames have to be consolidated to output the results of a single well. Two techniques have been considered to consolidate the results of the whole well namely, stitching and overlap-imaging (block 206). Stitching of the image frames requires overlap imaging of 5-20% of the size of the frames. In the illustrated system, the beads are of specific dimension of 30×110 μm.

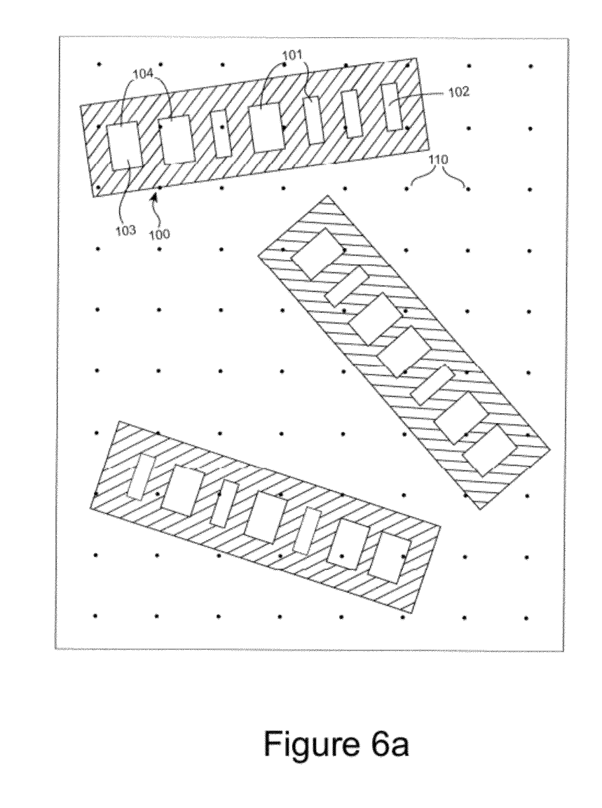

One preferred configuration is to take a large image (for example, 6-8 mm in diameter of a microwell) with sufficient optical resolution to resolve the barcode pattern (5 pm and 10 pm). If a 4× objective is used, more area can be covered. However, spatial resolution is not as good as when a 10× objective is used. If a 10× objective is used for a microwell with a diameter of 6.0-7.0 mm, it needs to be indexed to scan 5×6 frames (FIG. 4(a), image frames FRij, where i 1 to 6, j=1 to 5). With an overlapping of frames, the beads at the periphery of the frames will fall in one of the adjacent frames. The largest dimension of the bead (110 pm) and a safety margin of 10 pm are considered for overlapping. Therefore the whole well bottom is imaged with an overlapping of 120 pm at the edges in both directions. If one of the beads with a barcode pattern happen to be located between the two image frames (see beads Ba between frames FRa and FRb, and beads Bb between frames FRb and FRc in FIG. 4(a)), each image frame FRa, FRb and FRc will have a partial head or partial barcode (5 pm×15 pm) image.

If two neighboring images after being patched are slightly off in either the X or Y direction, the barcode may not be recognizable and decoding will be difficult. Therefore, a novel bead image overlapping method is used. This method is to overlap each neighboring frame FRa, FRb and FRc with an overlap of 120 pm, for example, (the long axis of the bead+margin) on X and Y direction (FIG. 4(b)). The overlapping is made so that the beads are imaged in two images, or more if overlapped in both X and Y directions. Adjacent frames will “fall fully” in at least one of the frames (as illustrated in FIG. 4(c), beads Ba fall fully in frame FRb and beads Bb fall fully in frame FRc; part of beads Ba remain on frame FRa).

Image processing is carried out in parallel to the movement of the XY stage (block 208). The separate frames with bead patterns are sent to the image-processing module as soon as they are imaged (FIG. 4(d)). The image decoding process will only process whole beads (block 210), and do not need to process the partial beads remaining in any frame. By this method, perfect image patching is not necessary, and decoding is much faster and accurate. Each well is processed frame by frame within a specified time period (e.g., 1 minute) before the mechanical movement of the stage is complete. Image processing is carried out on the fly for each well before the next well is scanned. For each image frame, bead decoding is carried out based on the bead image using the grid search process as described above (block 212). It is noted that FIG. 4 illustrates overlaps of frame edges in one of X-Y directions. Overlaps of frame edges in the other one of X-Y directions is similar for the frames. Further, it is noted that the number of frames (mXn) required to cover the entire area of beads on the support (e.g., well bottom) would depend on the detection optics, individual frame size and overall support area.

### Image Processing Algorithm

As soon as a barcode image is obtained from the CCD, the image data is rapidly processed by the barcode decoding software. They are many implementations of the image-decoding algorithms. Depend on the image patterns, different algorithms may vary in terms of decoding speed or accuracy. For detection of the bars in beads, two different approaches may be implemented. One approach is to reconstruct the periphery of the bars by eroding and filling the images, using an approach based on tracking object similar to tracking the bead body discussed in the earlier embodiment. Another approach is to rely on a ‘grid search’ based routine which searches the bars in the beads in an image frame through a grid.

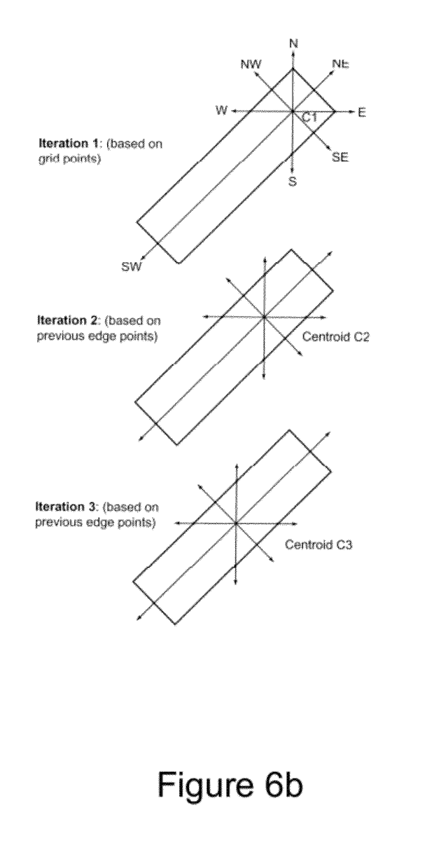

FIG. 2(c) illustrates an exemplary flowchart of a segmentation based algorithm 250. The algorithm includes five main sub-processes: (1) Enhancement of bright field image (block 260); (2) Grayscale threshold (block 280); (3) Segmentation and area filtering of bars (block 300); (4) Dilation and area filtering of beads (block 320); and (5) Pattern search and decoding of barcodes (block 340). Some of these processing are carried out using the mathematical software, such as toolkits available from “The Mathworks, Inc.” (e.g., MATLAB® Version 7.4.0.287 (R2007 a); Jan. 29, 2007), NI Machine Vision/Labview/NI Developer Suite 2009 and Visual Studio 2009 softwares. The functions of these processes are explained in the following sections and are shown in FIG. 5.

(1) Enhancement of image: The performance of the decoding of beads depends heavily upon the quality of the image. The accuracy of the decoding process can be improved by imaging enhancement, shown using exemplary flowchart 260 in FIG. 2(d). This image enhancement using image intensity normalization to provide uniform intensity background (block 262). Non-uniform background is often due to the non-homogeneous illumination. To achieve high image contrast of the beads, the background should be made homogeneous (block 264) first by background subtraction and normalization (block 266).

(2) Grayscale threshold: FIG. 2(e) is an exemplary flowchart 280 illustrating a possible grayscale processing routine. The bright field image of the beads has only two color components namely, black nickel bars and while bead body. However during imaging a continuous range of grayscale pixels are formed. The nickel bars can be separated from the bead body using grayscale thresholding, including calculating and adjusting a threshold factor (block 282), converting the threshold grayscale into a binary image (block 284) and filling holes in the image to limit the objects (block 286). The edge or border of the beads are also appeared in the grayscale image as noise which are removed in the successive steps of the processing.

(3) Segmentation and Area Filtering of bars: FIG. 2(f) is an exemplary flowchart 300 illustrating a routine for performing segmentation and area filter of bars. The bars in the beads are the only high contrast elements in the beads. So recognition of the beads is done by detecting the bars in the heads. The goal of image segmentation is to separate the bars in the image for further analysis. The seven bars of the beads including MSB, 5 bits and LSB are segmented in order to measure their dimension for decoding. The routine 300 includes image erosion to reduce a size of stray particles (block 302), filtering out stray edges or particles (block 304) and filtering based on a rectangle length (block 306). After the segmentation of the bars, any other objects present in the images are filtered out using the range of area of small and big bars (block 308).

(4). Dilation and Area Filtering of beads: FIG. 2(g) is an exemplary flowchart 300 illustrating a routine for performing dilation and area filtering of beads. After segmentation of the bars, each set of seven bars is considered a bead. The set of seven bars are merged together using dilation of the bars (block 322). The routine may also eliminate overlapping beads (block 324). After the bars are dilated the objects formed as beads are filtered out using the area of the beads with a tolerance (block 326). An additional filtering step may be included to remove the ring around the set of seven bars due to the edges of the beads.

(5). Pattern search and decoding of barcodes: FIG. 2(h) is an exemplary flowchart 340 illustrating a routine for performing a pattern search and decoding of barcodes. After the bead is found, the set of seven bars is matched with a pattern search. In order to decode the barcode, the widths of the transmission intensity peaks of the set of seven bars are analyzed. A half maximum line is used to calculate the widths of the peaks. In order to extract the binary bit information, 4-6 pixels are used to describe the narrow bar (‘0’) of the beads and 9-11 pixels are used to describe the narrow bar (‘I’) of the beads. Based on the ratio of the width of the bars and their adjacent spacing on the right, the digits are quantitatively decoded.

In other words, the routine 340 may include calculating the orientation of the bead (block 342), averaging the pixel intensity along the bead length (block 344), calculating the bars and spaces array within a bead (block 346), computing a ratio between bars and spaces (block 348), decoding the ratios 1.5-2.5 as ‘1’ and .r-1.4 as ‘0’ (block 350), extracting indices of a ‘path’ around the bead (block 352) and averaging the fluorescent intensity from the fluorescent image (block 354).

In order to reduce the amount of computational time for the image processing, a number of attempts have been made to improve the algorithm. The image resolution is reduced from 1 p.m/pixel to 10 μm/pixel in order to do the initial processing for segmentation of the beads. When the beads are segmented, the barcodes are extracted using the high resolution image. Another attempt is to convert the image decoding algorithm from MATLAe to C and pre-compile the C program before execution. This tremendously improves the speed of execution of the image decoding software. Finally the decoding program is run with a co-processor such as NVIDIA and using a CUDA library to couple the program with the co-processor. This system has as many as 128 co-processors to execute the program in a parallel fashion.

Two different image processing approaches for decoding the “positive” and negative barcode beads may be implemented. One is based on object tracking of the bars of the beads in case of positive barcode beads and the other is based on detecting the body of the beads in the case of negative barcode beads. The object tracking based on detecting the set of bars is described earlier. The method of tracking the bead body is for the negative beads where the bead is detected using the boundary of the beads. The image processing steps involves a combination of routines such as dilation, erosion, edge detection, mask creation.

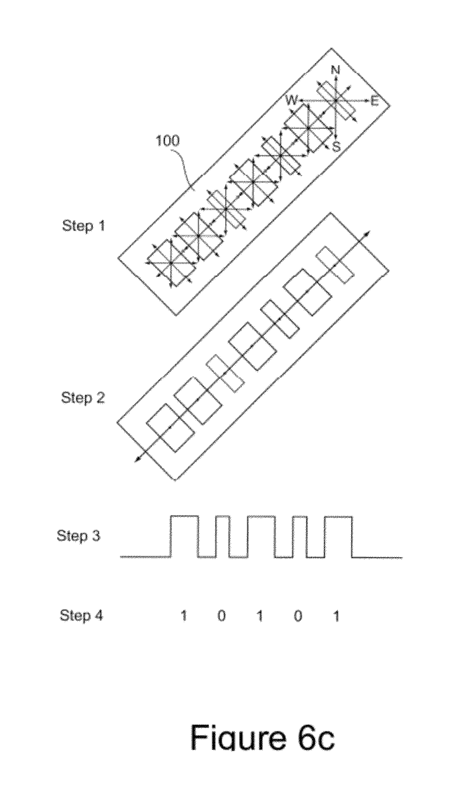

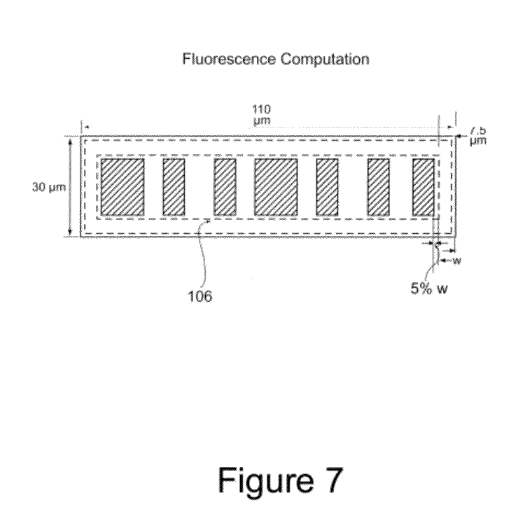

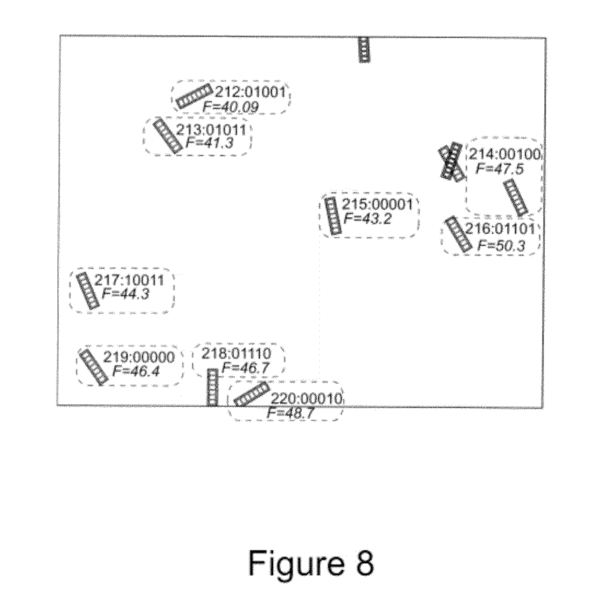

Grid search algorithm for barcode decoding: Grid search routine in and by itself is a known technique, which with the disclosure herein, can be effectively applied for bead decoding. The grid search routine used for decoding the beads discretizes the image frame into grids on x and y directions as shown in FIG. 6a. (In FIG. 6a, for purpose of ease of illustration only, the opaque bars are shown in white, and the transparent background of the bead is shown cross-hatched.) The grid pitch is a function of the smallest dimension of the beads. In this case of rectangular beads 100, the grid pitch is half the width of the beads (or 15 in the illustrated example). Accordingly, at least one of the grid points 110 falls on the bead area. Using the grid point 110, directional lines are drawn towards North, South, East, West, NE, SW, SE, NW directions, reaching the edges of the bead 100 (see also FIG. 6b). The points where the directional lines intersect the edges of the head 100 are averaged to estimate the location of the geometrical centre of the rectangular bead. This initial estimated center location is refined by several iterations, each with similar extending directional lines from the center (e.g., C1) found in the immediate preceding iteration, to obtain a new estimated center location (e.g., C2). After several iterations of center points, the geometrical center (e.g., centroid C3) may be determined at convergence (or equilibrium) of the estimates, and the major axis of the bead can also be determined. Using the major axis of the bead, decoding of the bead by decoding the barcode therein can be simply accomplished. The bars are all aligned along the major axis of the bead, and the sequence of wide bar 104 and narrow bar 101 can be determined from the image, with the MSB and LSB as reference points for the barcode.

Alternatively, the above grid search routine can be adapted for decoding the set of bars directly. In this case the grid search algorithm searches for the bars at each grid points and construct the contour of the bars in order to decode the bead. The grid pitch is half the width of the smallest bars/slits. In the illustrated example, the grid pitch is 2.5 1.1M.

There are four major steps in the grid search algorithm (shown in FIG. 6c) as follows:

1. Establish a rectangular search grid and search at each grid point for a bar.

2. Determine the axis of the bars from the centroids of the bars.

3. Determine the intensity profile of the bars of the beads.

4. Compute the overall bar code based on the sequence of bars determined above.

### Fluorescence Detection

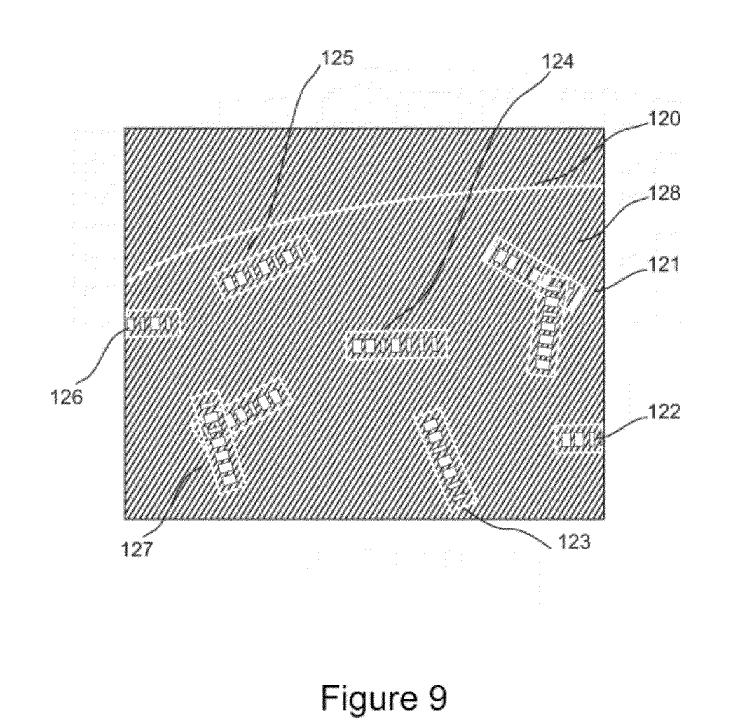

Fluorescence of the beads is calculated using the area between the two concentric rectangles in dotted lines illustrated in FIG. 7. The principle is based on quantifying the fluorescence along the path outside the barcode but inside the bead. This is the periphery region 106, which is 5% inside of the edge of the bead and 5% outside the central slit region 105 of the bead. The width of the path is pw, constant around the periphery of the bead. In this area there is no influence of the barcode metal surface on the reflection of fluorescence light. The average fluorescent value is calculated for the pixels along the path area of the beads in the fluorescence image. The path area pixel indices extracted from the bright field images are used for fluorescence calculation.

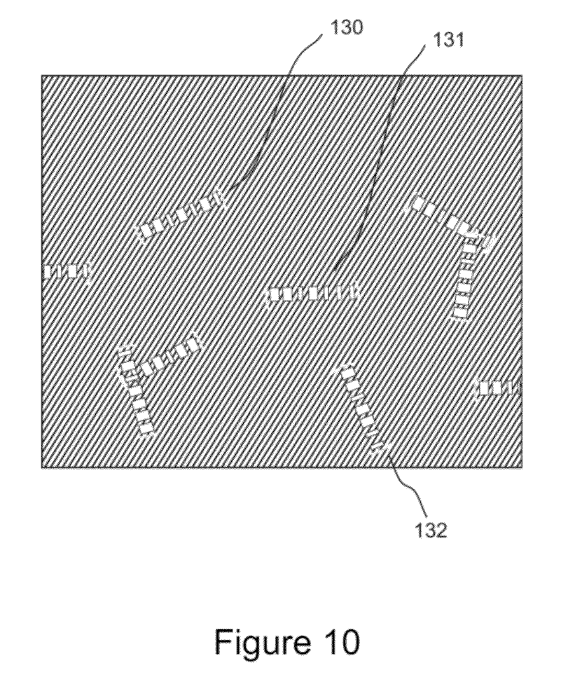

As was in the case of the earlier embodiment, the surface of the bead is prepared to provide a probe surface that can immobilize, hybridize, react and/or bond with a target sample carrying a fluorophore. The planar surface of the bead is continuous and uniform, without surface pits. However, at the opaque metal region, the fluorescence intensity is not uniform and so the fluorescence region 106 (path region) is the region from which the result of molecular immobilization is detected, even though immobilization takes place over the entire planar surface of the bead including the central bar region 105. By integrating the total fluorescence signal obtained from the area 106, confined within the dash line, as shown in FIG. 7, constant fluorescence value can be achieved. FIG. 8 is an exemplary image of beads in a single image frame. The bars in each bead appear dark on a light background (i.e., the “path” region for detected fluorescence). The image is imposed with data from bead decoding and fluorescence detection. In the image, the barcode on the beads, the delineation of the fluorescence region and the values from fluorescence calculation are shown as “F=” numbers.

The strategy for the image processing for different types of bead designs is summarized in the following.

1. Negative Beads without Border

Negative beads are beads with black background and transparent bars. Metal is present everywhere in the beads except on the code bars. The code bars are also called slits or window. Since metal is present all the way to the edge, these beads are black all the way to the edge. Image processing is more difficult in these beads because is difficult to separate out touching beads and watershed algorithm is deployed.

In accordance with this embodiment, a watershed algorithm in Matlab is applied to isolate the beads. Because the higher density of black pixels (due to opaque area) correspond to edges of the beads, the watershed transform finds ridgelines in an image and treat the surfaces enclosed by dense pixels as beads. Normally the beads have constant area and therefore each bead is separated from the image after filtering using their areas. In addition, the beads are recognized based on the slits (bars) present in the beads. The outline of the slits set is extracted using structure element transformation and filtration. With the good clarity of the slits, any noise in the background of the image is removed. The watershed algorithm in Matlab works for black and white images and so the image is first converted to black and white image.

### 2. Positive Beads

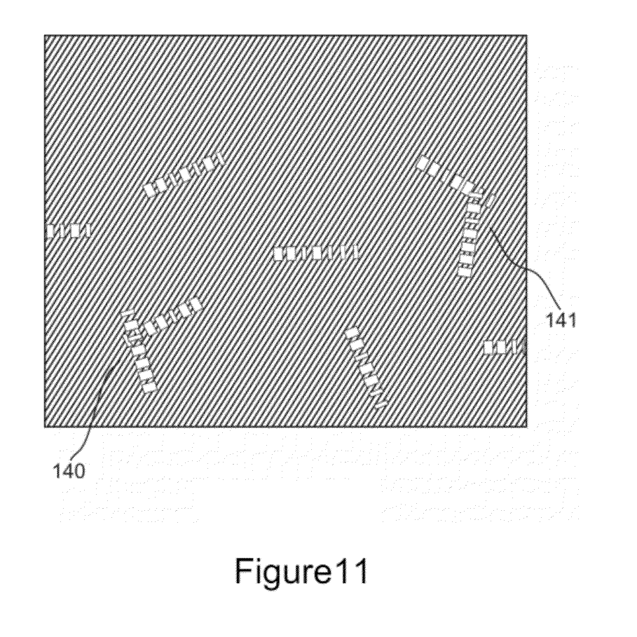

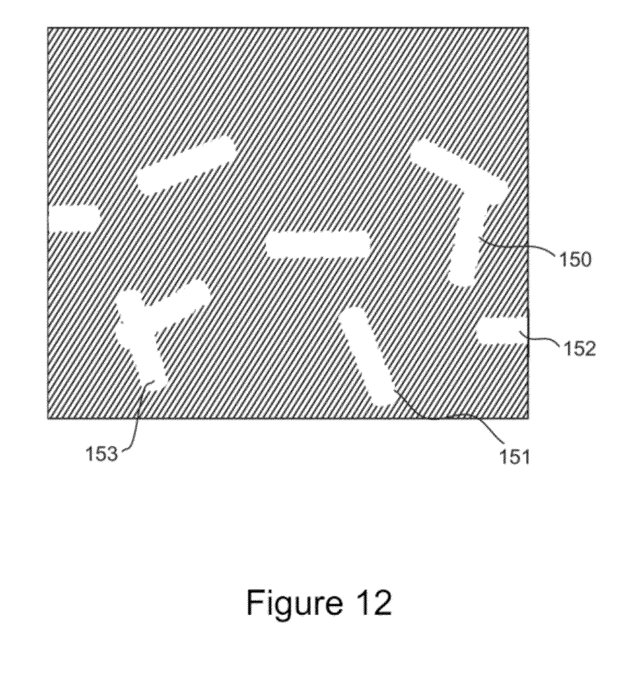

In these positive beads, the metal bars are encapsulated in photopolymer and so they appear with transparent background. The image processing steps presented earlier are suitable for these beads. The barcode decoding from a processed image could also be done, for example, using a geometry pattern recognition of the barcodes as illustrated in the following set of six figures (FIGS. 9-14) corresponding to six high level steps.

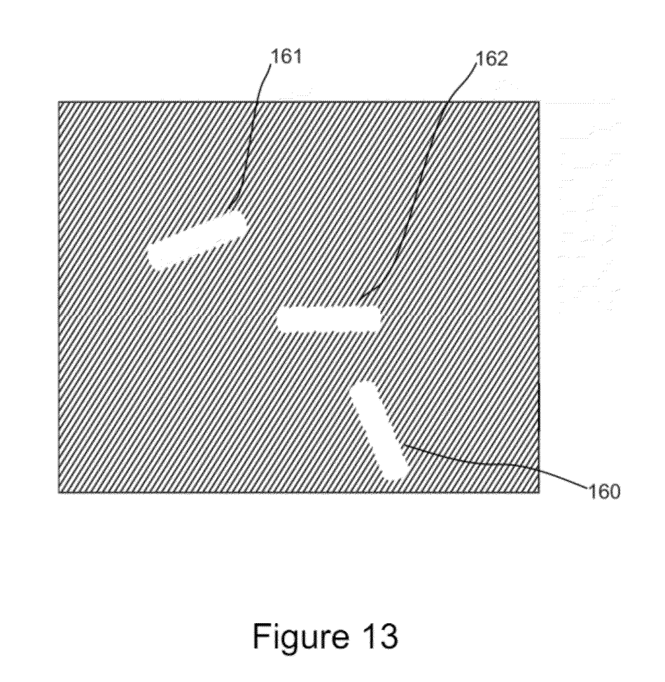

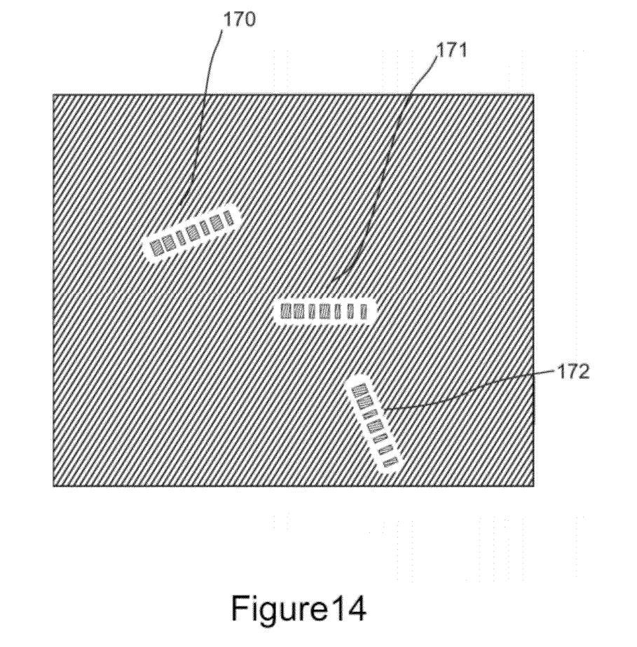

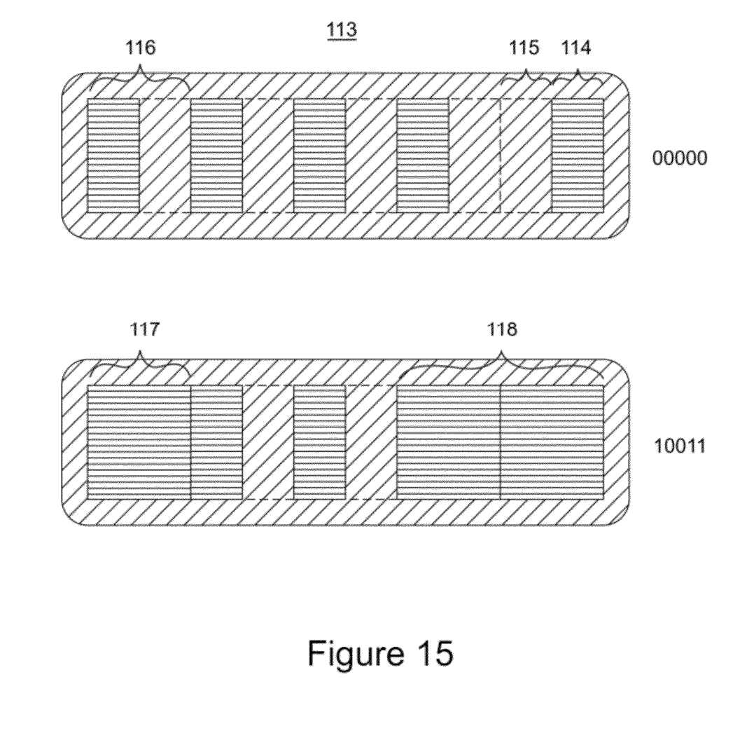

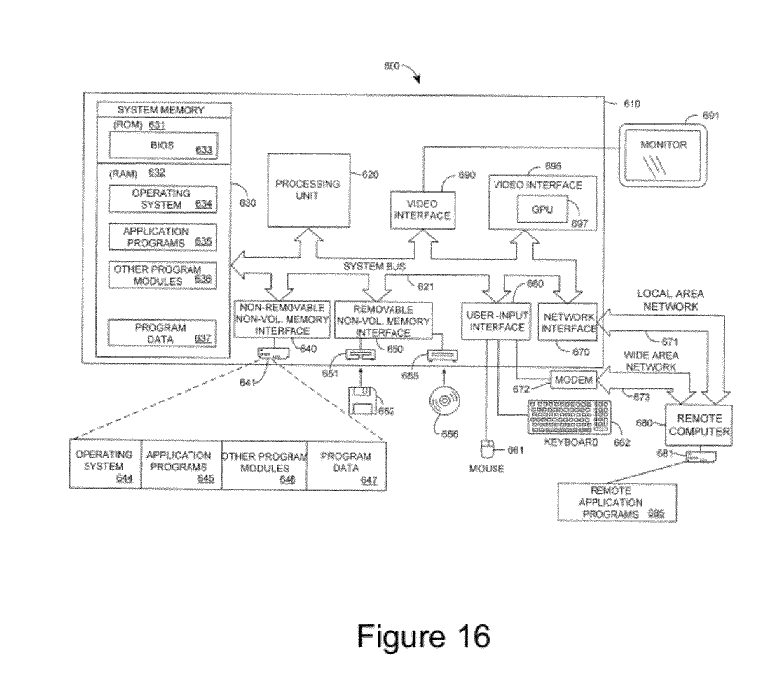

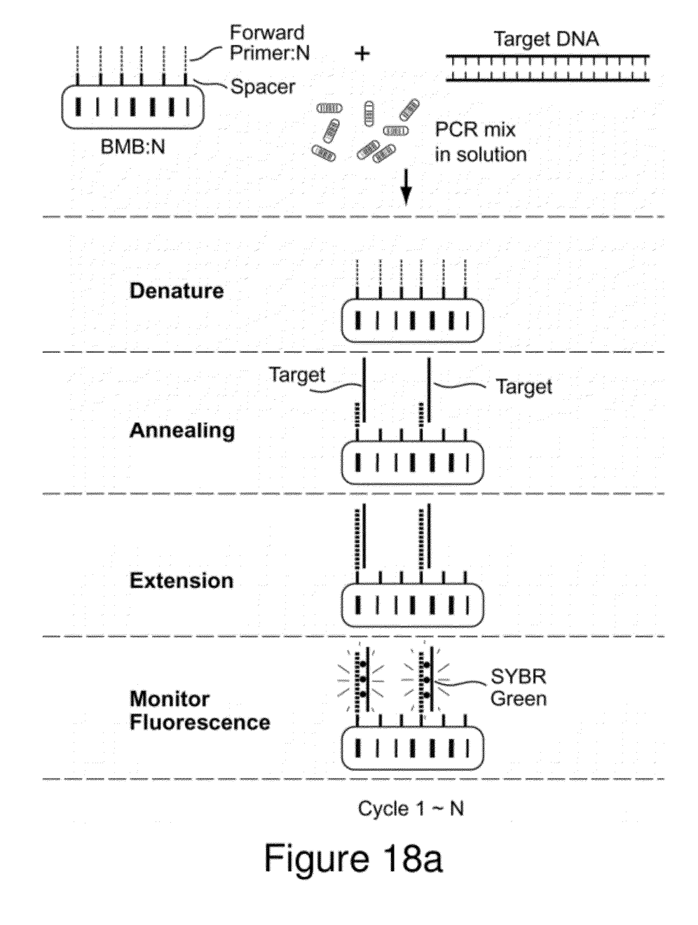

In a first step (FIG. 9) the gray scale images are undergone background correction and binary thresholding. The accuracy of the decoding process can be improved by imaging enhancement and normalization. Binary thresholding is needed for data reduction in order to carry out the image processing at higher speed. The edge of the well (120) is still visible in this image. In this image example, overlapping beads (121,127), partial beads (122, 126) and good beads (123, 124, 125) are shown in black background (128). In the second step (FIG. 16), the segmented images are filtered for the area of bars but the resultant image is left with a few stray patterns from the edge of the bead. The objects in the image with areas outside of the ranges of the areas of ‘0’ bars and ‘1’ bars will be removed. But still there will still be some edges (130, 131) on each end of the length of the bead with area that will fall in the range which may not have removed by this areal filtering. The third step (FIG. 11) is continued with filtering using a bounding box around the bead in order to remove edges on both side of the length of the bead. This process will completely remove all stray edges or objects in the image. But the overlapping edges (140, 141) will still remain in the image. In the fourth step (FIG. 12) the bars are dilated so that they collapse as a single object so that individual beads are converted in to objects (150, 151, 152). In this case there will still overlapping beads (150, 153) in the image. The fifth step (FIG. 13) filters the image with area of the bead so that partial beads or overlapping beads are removed from the image. The seven bars of the beads including MSB, 5 bits and LSB are segmented as good beads (160, 161, 162) in order to measure their dimension for decoding. In the final step (FIG. 14), the beads are matched geometrically using computer generated barcode patterns so that barcode detection is performed. The beads are decoded as 10101 (170), 101011 (171) and 101011 (172). Alternatively, the widths of the intensity peaks of the set of seven bars are analyzed. To decode the beads, 4-6 pixels are used to describe the narrow bar (‘0’) of the beads and 9-11 pixels are used to describe the narrow bar (‘1’) of the beads. Further, based on the ratio of the width of the bars and their adjacent spacing on the right, the digits are quantitatively decoded.

### 3. Surfboard Beads (Curved on Both Sides)

These beads are designed for flowing smoothly in microfluidic channels so that the barcodes can be read using spatial pixels in a line camera or temporally in a PMT. While adapting the same beads for image processing based multiplexed diagnostics platforms involve special image processing routines such as Hough transform and Sobel filter, in order to extend the decoding to curved rectangular shaped beads. Geometrically, the bead is composed of a rectangle in the middle and two semi circles on either ends. One of the semi circles is larger than the other and so they serve as MSB and LSB bits. In order to decode the barcodes, image processing is done to erode the bead except the semicircles. A filter based on area can extract only the MSB semicircle from the beads. This solid semi circle image is reduced to edged image using a ‘Sobel filter’. The whole image consists of a curve forming semicircle and a straight line. Using Hough transform in MATLAB® the straight lines are identified. Based on the straight-lines, boxes of lengths of the barcode portion of the beads are constructed towards the direction perpendicular to the straight lines but away from the semicircular regions. Extraction of the barcode is accomplished by plotting the intensity inside the constructed boxes.

### 4. Left Aligned Bars in Bead

The bar segments in the bead consists of a ‘transparent space’ component and a ‘black bar component. In one case the bar is aligned at the center of the bead segment so that the spaces are divided equally on either side of the bar. This is termed as centre aligned bars in bead. One advantage of left aligned bars system is that the decoding is done with higher accuracy because the spacing between barcode segments is constant. In this left aligned bars system, the bars are separated out in the bar segment and are placed on the left with space on the right. While decoding, the ratio between bars and space within a bar segment is computed to qualify as ‘1’ or ‘0’ bit.

5. Shortened Bead with No Spaces Between Code Segment

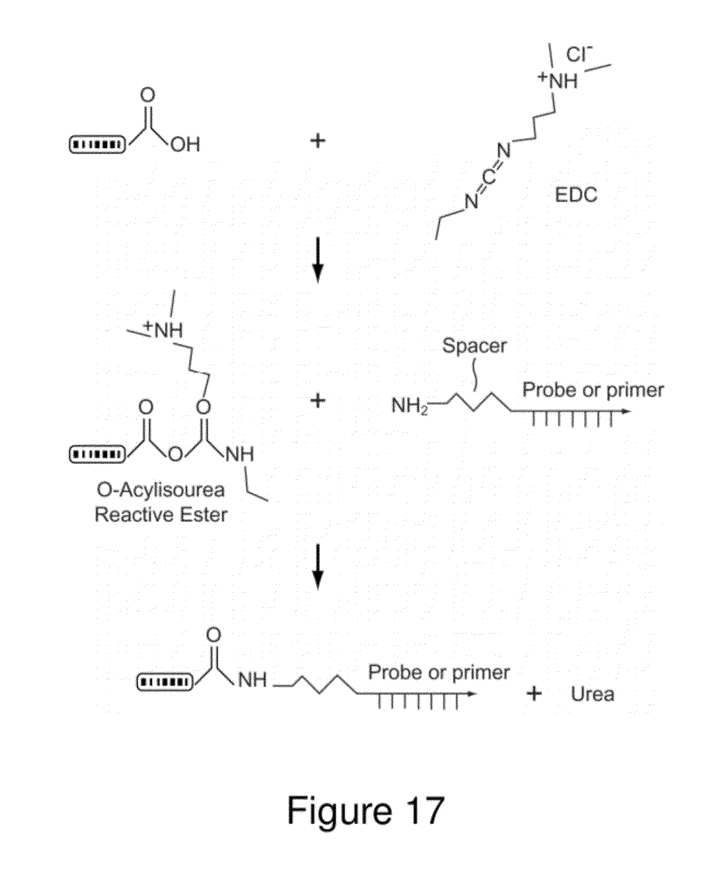

In order to reduce the length of the beads further, a 5 μm space that exists in between bar segments (in the previous design) are removed as shown in FIG. 15 In this case a bar segment consist of a 5 um bar (114) and 5 um space (113) to serve as ‘0’ bit (116) while bar segment with a 10 um bar serves as ‘1’ bit (117). In this bar-coding system, the bars merge for repeated ‘1’ bits (118). A border of less than 5 um (115) width helps in the decoding of the barcode in the case of touching beads. Further the border helps in the quantification of fluorescence. The MSB and LSB bits are further removed in this design while majority of the left aligned bar segments in the barcode accounts for MSB and LSB bits. Further the first bit is left aligned and the last bit is right aligned in order to keep the barcode length constant. The majority of the other bits will decide the direction of the barcode. If the majority of the bits read are right aligned then the barcode has to be reversed.

Referring again to the flowcharts described above, at least some of the blocks may be implemented utilizing the controller illustrated in FIG. 1. The controller may be part of a computer system, an example of which is illustrated in FIG. 16. FIG. 16 thus illustrates an example computing device in the form of a computer 610 that may be used to process the images discussed above.

Components of the computer 610 may include, but are not limited to a processing unit 620, a system memory 630, and a system bus 621 that couples various system components including the system memory to the processing unit 620. The system bus 621 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 610 typically includes a variety of computer readable media. Computer readable media can be any available media that can be accessed by computer 610 and includes both volatile and nonvolatile media, removable and non-removable media. By way of example, and not limitation, computer readable media may comprise computer storage media and communication media. Computer storage media includes volatile and nonvolatile, removable and non-removable media implemented in any method or technology for storage of information such as computer readable instructions, data structures, program modules or other data. Computer storage media includes, but is not limited to, RAM, ROM, EEPROM, FLASH memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 610. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, radio frequency, infrared and other wireless media. Combinations of any of the above should also be included within the scope of computer readable media.

The system memory 630 includes computer storage media in the form of volatile and/or nonvolatile memory such as read only memory (ROM) 631 and random access memory (RAM) 632. A basic input/output system 633 (BIOS), containing the basic routines that help to transfer information between elements within computer 610, such as during start-up, is typically stored in ROM 631. RAM 632 typically contains data and/or program modules that are immediately accessible to and/or presently being operated on by processing unit 620. By way of example, and not limitation, FIG. 16 illustrates operating system 634, application programs 635, other program modules 636, and program data 637.

The computer 610 may also include other removable/non-removable, volatile/nonvolatile computer storage media. By way of example only, FIG. 16 illustrates a hard disk drive 641 that reads from or writes to non-removable, nonvolatile magnetic media, a magnetic disk drive 651 that reads from or writes to a removable, nonvolatile magnetic disk 652, and an optical disk drive 655 that reads from or writes to a removable, nonvolatile optical disk 656 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 641 is typically connected to the system bus 621 through a non-removable memory interface such as interface 640, and magnetic disk drive 651 and optical disk drive 655 are typically connected to the system bus 621 by a removable memory interface, such as interface 650.

The drives and their associated computer storage media discussed above and illustrated in FIG. 16, provide storage of computer readable instructions, data structures, program modules and other data for the computer 610. In FIG. 16, for example, hard disk drive 641 is illustrated as storing operating system 644, application programs 645, other program modules 646, and program data 647. Note that these components can either be the same as or different from operating system 634, application programs 635, other program modules 636, and program data 637. Operating system 644, application programs 645, other program modules 646, and program data 647 are given different numbers here to illustrate that, at a minimum, they are different copies. A user may enter commands and information into the computer 20 through input devices such as a keyboard 662 and cursor control device 661, commonly referred to as a mouse, trackball or touch pad. A camera 663, such as web camera (webcam), may capture and input pictures of an environment associated with the computer 610, such as providing pictures of users. The webcam 663 may capture pictures on demand, for example, when instructed by a user, or may take pictures periodically under the control of the computer 610. Other input devices (not shown) may include a microphone, joystick, game pad, satellite dish, scanner, or the like. These and other input devices are often connected to the processing unit 620 through an input interface 660 that is coupled to the system bus, but may be connected by other interface and bus structures, such as a parallel port, game port or a universal serial bus (USB). A monitor 691 or other type of display device is also connected to the system bus 621 via an interface, such as a graphics controller 690. An additional graphics controller 695 with may be connected to the system 621, where the GPU 697 of the additional graphics controller 695 may be used, in conjunction with one or more virtual machines and a suitable mechanism for offloading floating point calculations to the GPU 697, such as Compute Unified Device Architecture (CUDA), Open Computing Language (OpenCL).

The computer 610 may operate in a networked environment using logical connections to one or more remote computers, such as a remote computer 680. The remote computer 680 may be a personal computer, a server, a router, a network PC, a peer device or other common network node, and typically includes many or all of the elements described above relative to the computer 610, although only a memory storage device 681 has been illustrated in FIG. 16. The logical connections depicted in FIG. 16 include a local area network (LAN) 671 and a wide area network (WAN) 673, but may also include other networks. Such networking environments are commonplace in offices, enterprise-wide computer networks, intranets and the Internet.

When used in a LAN networking environment, the computer 610 is connected to the LAN 671 through a network interface or adapter 670. When used in a WAN networking environment, the computer 610 typically includes a modem 672 or other means for establishing communications over the WAN 673, such as the Internet. The modem 672, which may be internal or external, may be connected to the system bus 621 via the input interface 660, or other appropriate mechanism. In a networked environment, program modules depicted relative to the computer 610, or portions thereof, may be stored in the remote memory storage device. By way of example, and not limitation, FIG. 16 illustrates remote application programs 685 as residing on memory device 681.

The communications connections 670, 672 allow the device to communicate with other devices. The communications connections 670, 672 are an example of communication media. The communication media typically embodies computer readable instructions, data structures, program modules or other data in a modulated data signal such as a carrier wave or other transport mechanism and includes any information delivery media. A “modulated data signal” may be a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Computer readable media may include both storage media and communication media.

### Polymerase Chain Reaction (PCR)

The polymerase chain reaction (PCR) is a technique in molecular biology to amplify a single or few copies of a piece of DNA across several orders of magnitude, generating thousands to millions of copies of a particular DNA sequence. The method relies on thermal cycling, consisting of cycles of repeated heating and cooling of the reaction for DNA melting and enzymatic replication of the DNA. Primers (short DNA fragments) containing sequences complementary to the target region along with a DNA polymerase (after which the method is named) are key components to enable selective and repeated amplification. As PCR progresses, the DNA generated is itself used as a template for replication, setting in motion a chain reaction in which the DNA template is exponentially amplified. The vast majority of PCR methods use thermal cycling, i.e., alternately heating and cooling the PCR sample to a defined series of temperature steps. These thermal cycling steps are necessary first to physically separate the two strands in a DNA double helix at a high temperature in a process called DNA melting.

At a lower temperature, each strand is then used as the template in DNA synthesis by the DNA polymerase to selectively amplify the target DNA. The selectivity of PCR results from the use of primers that are complementary to the DNA region targeted for amplification under specific thermal cycling conditions. At the start of a PCR reaction, reagents are in excess, template and product are at low enough concentrations that product renaturation does not compete with primer binding, and amplification proceeds at a constant, exponential rate. Exactly when the reaction rate ceases to be exponential and enters a linear phase of amplification is extremely variable, even among replicate samples, but it appears to be primarily due to product renaturation competing with primer binding. At some later cycle the amplification rate drops to near zero (plateaus), and little more products is made.

Real-time PCR assays used for quantitative reverse transcription PCR(RT-PCR) combine the best attributes of both relative and competitive (end-point) RT-PCR in that they are accurate, precise, capable of high throughput, and relatively easy to perform. For the sake of accuracy and precision, it is necessary to collect quantitative data at a point in which every sample is in the exponential phase of amplification. Analysis of reactions during exponential phase at a given cycle number should theoretically provide several orders of magnitude of dynamic range. Rare targets will probably be below the limit of detection, while abundant targets will be past the exponential phase. In practice, a dynamic range of 2-3 logs can be quantitated during end-point relative RT-PCR. In order to extend this range, replicate reactions may be performed for a greater or lesser number of cycles, so that all of the samples can be analyzed in the exponential phase. Real-time PCR automates this otherwise laborious process by quantitating reaction products for each sample in every cycle. The result is an amazingly broad 107-fold dynamic range, with no user intervention or replicates required. Data analysis, including standard curve generation and copy number calculation, is performed automatically. As more labs and core facilities acquire the instrumentation required for real-time analysis, this technique may become the dominant RT-PCR-based quantitation technique.

### Encoded Microbead Based Multiplex Bioassay Protocol

Encoded microbead-based multiplex bioassays are very flexible, and can be easily adapted for existing real-time PCR or other amplification protocols. The following sections describe how to couple the oligonucleotide onto the encoded microbead and some examples of how to using encoded microbeads for multiplex amplification assays.

### Coupling Oligonucleotide to Encoded Microbeads

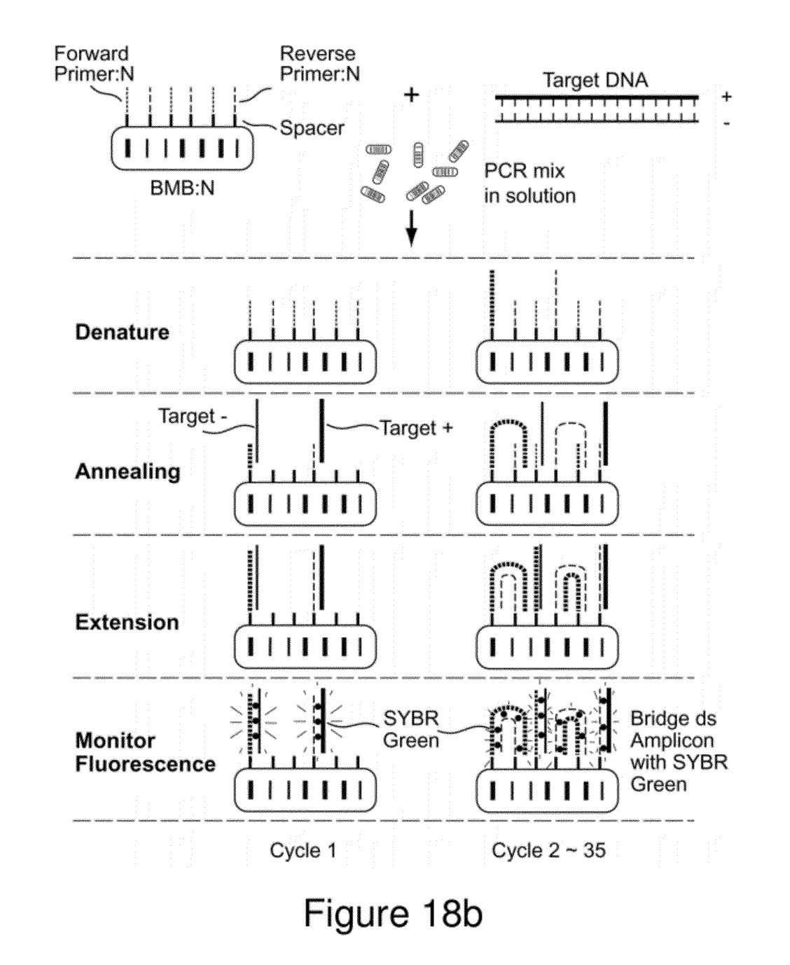

FIG. 17 illustrated the procedure of coupling aminoC6 modified oligonucleotide to carboxyl microbeads with a specific barcode N. A general protocol for coupling oligonucleotide to carboxyl microbeads is described here:
(1): Type N(N=1-128 barcode types) encoded microbeads
(2): Carboxyl group covalently attached on encoded microbeads.
(3): Amino group attached to 5′ or 3′ of oligonucleotide with C6-C12 spacer
(4): Oligonucleotide with Amine modified on 3′ or 5′ end, and will be coupled to carboxyl encoded microbeads. These oligo will be used as primers or probes for on encoded microbeads target amplification and detection.
(5): With the presence of EDC, first it reacts with carboxyl group and forms an amine-reactive O-acylisourea intermediate, and then quickly reacts with amino group on oligo to form an amide bond and release of an isourea by-product.

Materials for Coupling Process:

1. 25 mM MES Buffer, pH 4.0-6.0

2. 25 mM MES-T Buffer with 0.01% Tween 20, pH 4.0-6.0

3. Tris-HCl 50 mM, pH 7.4

4. 1×PBS (pH 7.4)

5. 1×PBS/1% BSA

6. 1×PBS-T (Tween-20, 0.05%-0.1%)

7. nuclease-free Water

8. EDC (Cat #: c1100−3×10 mg, ProteoChem, Denver, Colo.)

9. Amino C6-C12 modified oligonucleotide (100 μM in nuclease-free Water)

10. Carboxyl BMBs

Equipments used in Coupling Process:

1. BioShake XP (QInstruments)

2. Magnetic stand

3. Vortex

Experiment Procedure in Coupling Process:

Amino Modified Oligo Coupling to Carboxyl Microbeads:
1. Take up to 500 k BMBs from stock solution into a new 2 ml tube
2. Wash BMBs twice with MES-T buffer by vortexing the microbeads for 10 seconds; spin down for 10 seconds, and hold the microbeads in tube using magnetic stand. Remove the supernatant.
3. Add 159 μl MES-T buffer to the beads, vortex 5 second.
4. Add 1 μl oligonucleotide (0.5 μM final in 200 μl. Note: oligo concentration could be varied from 12.5 μM to 0.05 μM), vortex 5 second, and quick spin to bring the BMBs down to the bottom of tube.
5. Shaking at RT for 5-10 min (e.g. 1600 rpm using Bio Shake XP), while preparing EDC 10 mg in 1 ml of cold 25 mM MES buffer.
6. Immediately add 40 μl of fresh prepared EDC solution, vortex for 5 second, quick spin to bring the microbeads down, and then incubate for 2 hrs at RT, with shaking at 1600 rpm.
7. Remove the supernatant, and treat the microbeads with 500 μl Tris-HCl 50 mM, pH 7.4 for 15 min at RT, with shaking at 1600 rpm
8. Wash the microbeads once with 500 μl PBS/1% BSA buffer pH 7.4 as in step 2.
9. Block samples with 500 μl PBS/1% BSA: incubate for 1 hr at RT with shaking at 1600 rpm.
10. Remove the blocking buffer. Resuspend the beads in 5000-1000 μl PBS-T. Count the beads, and store the beads at 4° C., or process for PCR reaction.

Target amplification by PCR/RT-PCR or real-time PCR/RT-PCR is widely used method for target identification, and many different method exist. These methods could be easily adapted to encoded microbeads based target amplification with advantage of much higher multiplicity than conventional PCR/RT-PCR methods, especially for real-time PCR/RT-PCR methods. Here some methods adapted to encoded microbead technology are described.

### End-point Multiplex PCR on Encoded Microbeads

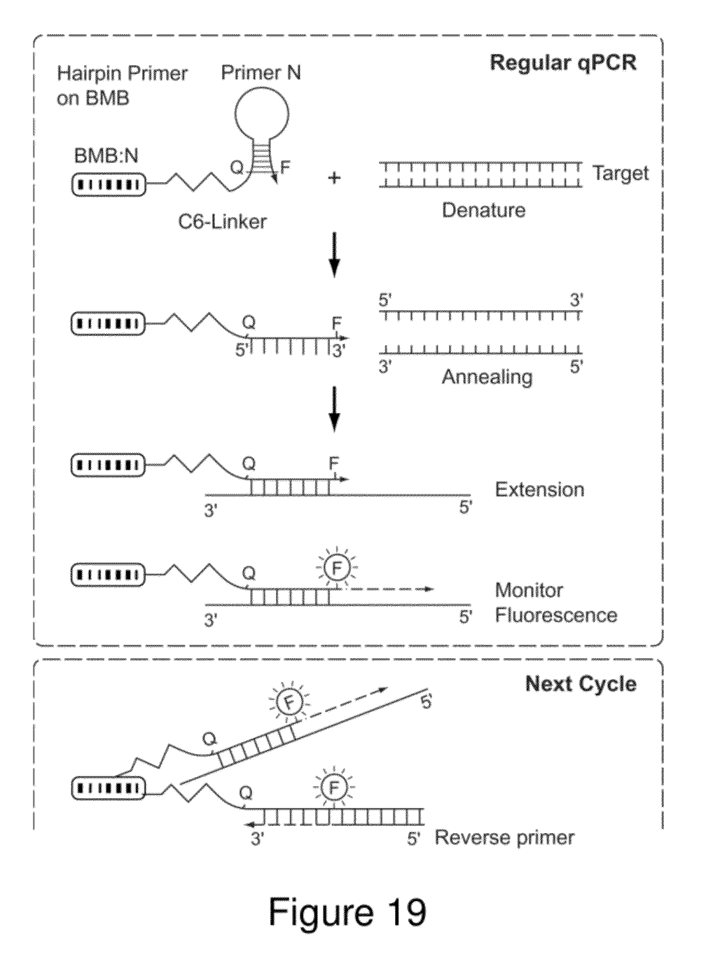

FIG. 18 illustrated the major steps for a solid phase PCR amplification and detection on labeled microbeads. For signal detection, SYBR Green could be used for detecting double stranded amplicon formed on labeled microbeads.

The soluble forward primer could be the same as the forward primer coupled on labeled microbeads. The soluble forward primers are present at a concentration less than that of their reverse primer counterpart (the ratio could vary from 1:1.5 to 1:16). In some condition, the PCR amplicon can be generated between the forward primer coupled on labeled microbeads and reverse primers in solution.

Asymmetric PCR reactions will take place in liquid phase. This will generate many single stranded PCR products.

During the asymmetric amplification in the liquid phase the produced amplicons will recognize the solid phase forward primers and anneal to these. The solid phase forward primers could be the same as soluble forward primers, or nested forward primers. These newly formed hybrids act as substrates for the polymerase in the same way as the liquid phase hybrids. As a result the solid phase primers will be elongated using the annealed products as templates. The elongated products remain attached to the labeled microbeads via the covalently bound solid phase primers.

At the end of PCR reaction, the sample plate will be removed, and the labeled microbeads will be washed with PBST buffer, and the plate will be scanned with Biocode Analyzer.

If the reverse primers were labeled with biotin, an extra step of SA-PE treatment will be processed: The washed labeled microbeads will be incubated with SA-PE solution for 10 minutes at room temperature with shaking. The SA-PE treated labeled microbeads will be washed again with PBST buffer and the plate will be scanned with Biocode Analyzer.

Protocol for on labeled microbeads PCR or RT-PCR Target Amplification, and Signal Detection by Streptavidin-R-PE:

Materials for Target Amplification and Signal Detection:
1. 1×PBST (Tween-20, 0.1%)
2. nuclease-free Water
3. labeled microbeads with Forward Primer conjugated.
4. 5′ Biotin Labeled Reverse Primer: 10 pM/μl, 5′-Biotin modified-MWG-Biotech AG
5. Streptavidin-R-Phycoerthrin Conjugate (SA-PE) (1 mg/ml)-Invitrogen
6. hybridization buffer: TMAC 3M, Sarkosyl 0.1%, Tris-HCl 50 mM pH8.0, EDTA 4 mM pH8.0
7. Qiagen Multiplex PCR Kit: Cat #: 206143 or 206145
8. Qiagen One-Step RT-PCR Kit: Cat #: 210210 or 210212

### Experiment Procedure:

On Labeled Microbeads PCR/RT-PCR Reaction:
1. Prepare a reaction mix according to table below. The reaction mix contains all the components required for PCR/RT-PCR except the template DNA/RNA. For labeled microbeads preparation, take enough labeled microbeads into 1.5 ml tube. Remove the PBST buffer, and then add nuclease free H₂O and all component to labeled microbeads except DNA/RNA template. Mix well and aliquot appropriate volume into PCR tubes.
2. Add 2 μl template DNA/RNA per PCR tube
3. Place the PCR tubes in the thermal cycler and start the cycling program as outlined in table below. The signal will be detected at each annealing step.

| | | Final |
|---|---|---|
| Component for multiplex PCR | 1 reaction | Concentration |
| Forward primer coupled labeled microbeads | 16 μl | |
| (50 beads per type) in H₂O | | |
| 2x PCR Mix | 25 μl | 1x |
| Reverse primer (10 μM) | 2 μl | 0.05-0.4 μM |
| 5x Q Solution | 5 μl | 0.5x |
| DNA sample | 2 μl | |
| Total volume | 50 μl | |

| Component for OneStep RT-PCR | 1 reaction | Final Concentration |
|---|---|---|
| Forward primer coupled labeled | 21 μl | |
| microbeads (50 beads per type) in H₂O | | |
| 5x OneStep RT-PCR buffer | 10 μl | 1x |
| dNTP Mix (10 mM of each dNTP) | 2 μl | 400 μM each |
| Reverse primer (10 μM) | 3 μl | 0.6 μM |
| 5x Q Solution | 10 μl | 1x |
| OneStep RT-PCR Enzyme Mix | 2 μl | |
| Rnase inhibitor (optional) | variable | 5-10units/reaction |
| RNA template | variable | 1 pg-2 μg/reaction |
| Total volume | 50 μl | |

Notes: The soluble primers final concentration should be optimized (e.g. 0.1-0.6 μM). The ratio between biotin labeled reverse primer and soluble forward Primer is 10:1, but different ratio could be used, and need to be optimized.

PCR Reaction

| | | | | | |
|---|---|---|---|---|---|
| Initial activation step: | | | 15 | min | 95° C. |
| Number of cycles: 30-45 | 3-step cycling: | 2-step cycling: | | | |
| | Denaturation: | Denaturation: | 30 | second | 94° C. |
| | Annealing: | Anneal/Extension: | 0.5-1 | min | 50-68° C. |
| | Extension: | | 0.5-1 | min | 72° C. |
| Final extension | | | 5-10 | min | 72° C. |

RT-PCR reaction

| Reverse transcription: | 30 min | | | 50° C. |
|---|---|---|---|---|
| Initial PCR activation step: | 15 min | | | 95° C. |
| Number of cycles: 30-45 | 3-step cycling: | 2-step cycling: | | |
| | Denaturation: | Denaturation: | 30 second | 94° C. |
| | Annealing: | Anneal/Extension: | 0.5-1 min | 50-68° C. |
| | Extension: | | 0.5-1 min | 72° C. |
| Final extension | | | 5-10 min | 72° C. |

Labeled Microbeads—Based Multiplex qPCR Bioassay Protocol

To carry out labeled microbeads—based real-time PCR, researchers have to choose not only what primers to design, but also what detection chemistry to use. In many cases these decisions will be influenced, if not determined, by the thermocycler that they have access to and the instrument's chemistry and dye compatibility. Since PCR on labeled microbeads is like solid phase PCR in solution, many solid phase PCR can be adapted on labeled microbeads: e.g. Bridge PCR (both forward and reverse primers are covalently linked to a solid-support surface), conventional solid phase PCR (asymmetric PCR is applied in the presence of solid support coupling primer with sequence matching one of the aqueous primers) and enhanced solid phase PCR (solid phase PCR can be improved by employing high Tm and nested solid support primer with optional application of a thermal ‘step’ to favour solid support priming) etc.

The following sections describe how to expand the multiplexity using labeled microbeads technology for real-time amplification assays. The following detection formats have proven to be particularly useful in connection with the present invention, but shall not be understood as limiting the inventive scope.

Target amplification by real-time PCR/RT-PCR is widely used method for target identification, and many different methods exist. The present invention adapts the use of labeled microbeads based target amplification with advantage of much higher multiplicity than conventional PCR/RT-PCR methods as set out in the examples below.

### Example 1

### Labeled Microbeads—Based Multiplex qPCR with ds DNA-Binding Dye as Reporter

All real-time PCR systems rely upon the detection and quantitation of a fluorescent reporter, the signal increase is proportionate to the amount of PCR product in a reaction. In the simplest and most economical format, that reporter is the double-strand DNA-specific dye SYBR® Green. SYBR Green binds double-stranded DNA, and upon excitation emits fluorescent light.

Labeled microbeads—based multiplex qPCR, for target N, the forward primer N, as shown in FIG. 18a, can be coupled to labeled microbeads with specific barcode: N, and leave the reverse primer in aqueous solution (or vice versa), forward primer N can be coupled to one labeled microbeads N. After the denaturing, annealing, extension, and dye-DNA binding processes, PCR product accumulates on the bead and fluorescence on bead increases accordantly. For signal detection, SYBR Green (or other ds DNA-binding dye e.g. Eva Green (Biotium)) are commonly used for detection of double stranded (ds) amplicon.

In the similar fashion: for target N, as shown in FIG. 18b, forward and reverse primer pair N can be both coupled to one labeled microbeads N. After the denaturing, annealing, extension, and dye-DNA binding processes, PCR product accumulates on the bead and fluorescence increases. For signal detection, SYBR Green (or other ds DNA-binding dye e.g. Eva Green (Biotium)) could be used for detection of bridge double stranded (ds) amplicon formed on encoded microbeads. After 30-50 cycles, most of the bead surface will be covered with SYBR binding bridge ds amplicons.

An increase in DNA product during PCR will lead to an increase in fluorescence intensity. Both fluorescence intensities will be measured and barcode will be decoded on every single bead at the end of each extension cycle, thus allowing DNA concentrations to be measured on a labeled microbead. The end-PCR melt curve analysis could also be done on labeled microbeads.

The labeled microbeads are also suited for microRNA (miRNA) multiplex qPCR (or real-time RT-PCR) using SYBR Green as reporter. The miRNA samples are poly (A) tailed and reverse transcripted (RT) into 1st strand cDNA using universal RT primer. The miRNA specific primer N is coupled to labeled microbeads type N. The PCR reaction using miRNA specific forward primer and the universal reverse primer generates double stranded amplicon on labeled microbeads surface which can be detected by ds DNA-binding reporter like SYBR Green.

The following protocol describes the detailed experimental procedure for multiplex real-time RT-PCR using SYBR Green I and labeled microbeads. The procedure begins with reverse transcription of total RNA. The cDNA is then used as template for real-time PCR with gene specific primers.

Reagents and equipments include (1) Carboxyl labeled microbeads, (2) Oligonucleotide Primers One or both of a primer pair are designed and selected to be covalently coupled to carboxyl labeled microbeads with a specific barcode. The 5′ aminoC6 modified primers (with or without oligo (dT)3-15 as spacer) need to be synthesized, (3) SYBR Green PCR master mix (e.g. Applied Biosystems; Qiagen etc.), (4) Optical flat-bottomed 96-well plate or strips (Applied Biosystems), and (5) SuperScript First-Strand Synthesis System for RT-PCR (Invitrogen).

### Detailed Procedure—Reverse Transcription

Reverse Transcription is carried out with the SuperScript First-Strand Synthesis System for RT-PCR. The following procedure is based on Invitrogen's protocol.

1. Prepare the following RNA/primer mixture in each tube:

| | |
|---|---|
| Total RNA | 5 μg |

Primer:

| | | |
|---|---|---|
| 50 μM oligo (dT)₂₀, or | 1 | μl |
| 2 μM gene-specific primer (GSP), or | | |
| 50 ng/μl random hexamers | | |
| 10 mM dNTP mix | 1 | μl |
| DEPC-treated H₂O | to 10 | μl |

2. Incubate the samples at 65° C. for 5 min and then on ice for at least 1 min.
3. Prepare reaction master mixture, adding each component in the indicated order. For each reaction: 10×RT buffer (2 μl), 25 mM MgCl2 (4 μl), 0.1 M DTT (2 μl), RNAaseOUT (1 μl), SuperScript™ III RT (200 U/μl) (1 μl).
4. Add 10 μl of reaction mixture to the RNA/primer mixture, mix gently, and collect by brief centrifugation.
5. Incubate as follows: Oligo (dT)₂O or GSP primed: 50 min at 50° C. Random hexamer primed: 10 min at 25° C., followed by 50 min at 50° C.
6. Terminate the reaction at 85° C. for 5 min. Chill on ice.
7. Add 1 μl RNase H and incubate at 37° C. for 20 min.
8. Store the 1st strand cDNA at −20° C. or use for real-time PCR immediately.

### Real-time PCR

1. If forward and reverse primer pair are both coupled on labeled microbeads, no soluble primer is needed. A working stock labeled microbeads could be prepared for each multiplex panel which includes 25-50 labeled microbeads for each target per samples. If only one primer (forward or reverse primer) is coupled on labeled microbeads, the other soluble primers for different targets are mixed to form 10× primer stock. Each primer concentration in the mixture is about 2 μM, and can be varied depending on each multiplex panel.

2. Set up the experiment and the following PCR program on the multiplex real-time PCR Instrument.

Three Step PCR program

| Cycles | Duration of cycle | Temperature (° C.) |
|---|---|---|
| 1 | 15 minutes | 95 |
| 30-50 | 30 seconds | 95 |
| | 1 minute | 55-65 |
| 1 | 5 minutes | 72 |

Two Step PCR program

| Cycles | Duration of cycle | Temperature (° C.) |
|---|---|---|
| 1 | 15 minutes | 95 |
| 30-50 | 30 seconds | 95 |
| | 3 minutes | 60-72 |
| 1 | 5 minutes | 72 |

3. Prepare the real-time PCR reaction mixture in each optical well. Each sample, standard and no cDNA control will be in duplicate or triplicate. Make sure the wells are free of bubbles
25-50 labeled microbeads per each target per sample per well. For 10 targets, a total of 250-500 labeled microbeads are used.
5 μl soluble primer mix (10× soluble multiplex primer mix if only forward or reverse primer coupled on labeled microbeads)
19.5-24.5 μl H₂O
25 μl SYBR Green Mix (2×)
0.5 μl cDNA (˜10-20 ng)
50 μl final

4. Instrument take readings of the amount of double stranded DNA amplified on each labeled microbeads in each well at each cycle and give the critical threshold (Ct) value, which represents the quantization of the product. The Ct is a relative value. This is why every experiment needs an internal control, usually a housekeeping gene.

5. After multiplex real-times PCR is finished, the instrument can perform dissociation curve analysis.

6. Analyze the real-time PCR result with multiplex real-time PCR software. Check to see if there is any bimodal dissociation curve or abnormal amplification plot.

Quantitative Real-Time PCR (qPCR)

For quantitative PCR, the plasmid DNA standard needs to be prepared.

### Procedures:

Plasmid DNA standard of the target gene:

1. Prepare the plasmid using the Qiagen plasmid prep kit.
2. Measure concentration of the plasmid DNA using Nanodrop.
3. Digest an aliquot of the plasmid DNA for linear DNA standard preparation.
4. Run an agarose gel of cut and uncut plasmid DNA to confirm the digest.
5. Prepare the linearized plasmid DNA standard stock of 10⁷ copy/μl (qPCR standard for the gene)
6. The concentration of the standard DNA is converted from ng/μl to copy number per μl using the following formula: (C * 10⁻⁹/MW)* N_{A} (C: standard DNA concentration: ng/μl, MW: molecular weight in Daltons (molecular weight of one bp=660), N_{A}: Avogadro's constant: 6.022×10²³)
7. Prepare a 10-fold dilution series with 7 steps (1-7) by diluting 20 μl of linearized plasmid DNA standard stock with 180 μl of TE. The resulting dilution series will span approximately 10⁶ copy/μl to 10° copy/μl of plasmid DNA.
8. Test the PCR reaction with this plasmid DNA dilution series.
9.

### Data Analysis:

Confirm that the negative control did not amplify. Calculate the linear regression of Ct versus log gene copy number in the plasmid DNA standards. Use this relationship to calculate the target copy number in the sample DNA from the Ct value for the sample.

### Example 2

### Labeled Microbeads—Based Multiplex qPCR/RT-PCR Using Hairpin Primers with Fluorophore

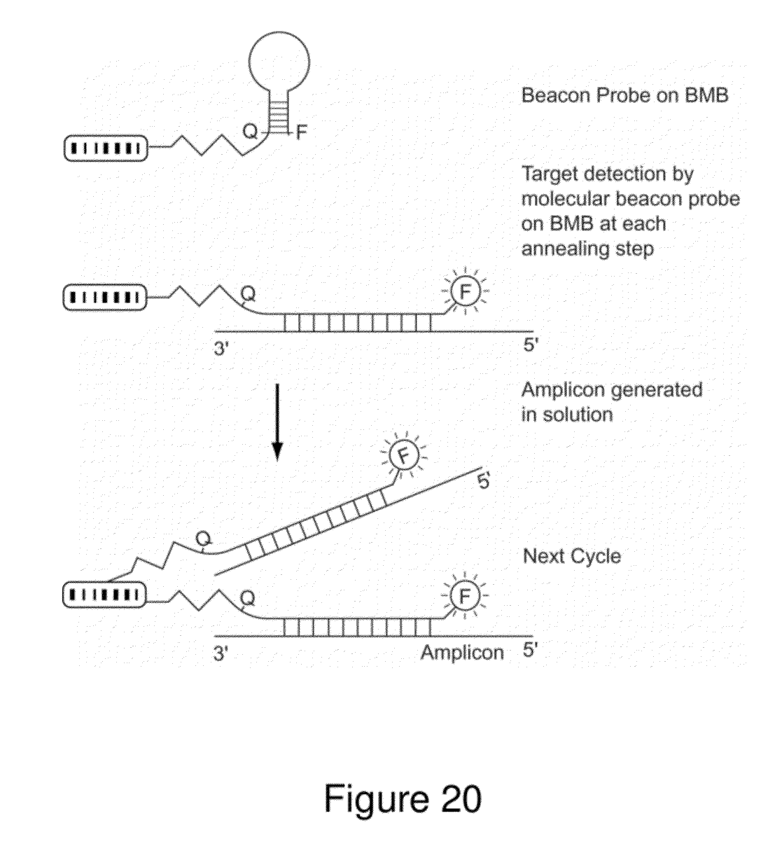

Hairpin primers consist of a single-stranded loop complementary to the target template and a tail of about 6 nucleotides are added to the 5′ end of the primer to form a blunt-end hairpin when the primer is not incorporated into a PCR product. A fluorophore is added on a base close to the 3′ end with (or without) quencher added at 5′ end. When the probe is in hairpin configuration, the fluorophore and quencher are in close proximity. Upon binding, the stem comes apart and the fluorophore and quencher are separated, giving off fluorescence. These probes are very flexible and can be used for any existing PCR application. FIG. 19 illustrates multiplex real-time PCR/RT-PCR: the major steps for real-time PCR amplification and detection on labeled microbeads. For labeled microbeads—based real-time PCR reaction, the forward hairpin primers, N, are coupled on labeled microbeads with a barcode number N. labeled microbeads will have abundance of primer molecules on its surface. At beginning, the labeled microbeads will not emit fluorescence from fluorophore F, because they are quenched by the quencher Q. If hairpin primer anneal with target sequence, and the fluorophore will be separated from quencher, and the signals will be detected on labeled microbeads. The double strand amplicon will be synthesized by PCR reaction on the bead surface; the fluorophore signal will be detected at the end of extension step of each cycle, and then denatured. After each cycle, the amount of amplicon is twice what it was before, and the fluorescence signal will increase accordantly.

The genomic DNA, or the 1st cDNA synthesized by reverse transcriptase can be used for multiplex targets amplification by on labeled microbeads PCR. To increase the amplification efficiency, the soluble forward primer without fluorophore and/or quencher could be added in solution with reverse primer. The forward primer's concentration could be less than reverse primer's concentration (1:1.5 to 1:16), so that the single strand DNA will be amplified, and will be served as template for forward primers coupled on labeled microbeads for the next cycle. The forward primers on labeled microbeads could have the same sequence as forward primers in solution phase. A nested target specific hairpin forward primer could also be designed to improve the detection specificity. The hairpin primer will be coupled to the carboxyl labeled microbeads by its 5′ aminoC6 linker leaving the 3′-end free to participate in PCR reaction

### Example 3

### Encoded Microbead-Based Multiplex qPCR/RT-PCR Using Molecular Beacons Probes

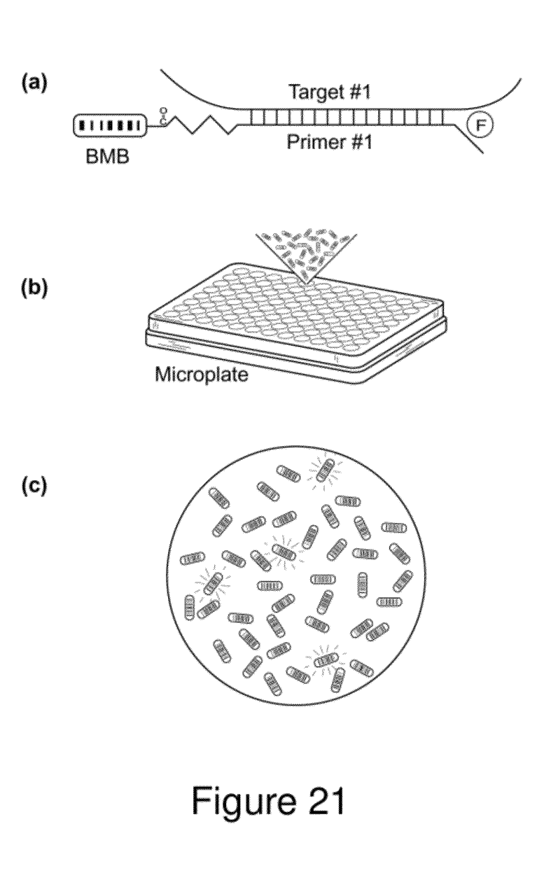

The principle of molecular beacon probe consist of a single-stranded loop complementary to the target template and a double-stranded stem, about six based in lengths, with a fluorophore at one end and a quencher at the other end, very similar to the hairpin primer illustrated in Example 2, but used as a probe molecule. FIG. 20 illustrated multiplex real-time PCR/RT-PCR on labeled microbeads by molecular beacons. The real-time PCR reaction can be performed with molecular beacon probe, N, coupled on labeled microbeads: N. labeled microbeads has abundance of probe molecules. Molecular beacons probes allow multiplex detection of PCR products. The signal will be generated only in the presence of the target, and remaining dark in its absence. The genomic DNA, or the 1st cDNA synthesized by reverse transcriptase will be used for multiplex targets amplification by asymmetric PCR. The primers could be designed for target specific amplification, or one or few pair of primer will be used for PCR amplification. Specific target identification will be detected by each specific molecular beacon probes coupled on different labeled microbeads after the annealing step. The forward primer's concentration is less than reverse primer's concentration (1:1.5 to 1:16) so that the single strand DNA will be amplified, and they will be served as template for hybridization with molecular beacons probes coupled on labeled microbeads. Once the molecular beacon probes bind to target amplicons, the quencher and fluorophore are separated, and the fluorophore signal will be detected at the end of annealing step of each cycle. After each cycle, the amount of amplicon is twice what it was before, and the fluorescence signal will increase accordantly.

### Example 4

### Labeled Microbeads—Based Multiplex Targets Identification by Isothermal Cleavase Reaction

Those of skill in the art can also use other DNA/RNA amplification techniques such as cleavase assay, and adapt them for real-time multiplex detection on encoded microbeads. The cleavase assay is an isothermal probe cycling, signal amplification reaction and is described in U.S. Pat. No. 6,913,881 and U.S. Pat. No. 7,011,944 the disclosures of which are hereby incorporated herein. The cleavase chemistry is composed of two simultaneous isothermal reactions. At primary reaction in solution, the 5′ flap probe is used for specifically and accurately detects single-base changes, insertions, deletions and changes in gene and chromosome number. At second reaction on labeled microbeads, the FREP probe is coupled on labeled microbeads, and is used for signal amplification and generic readout. Each 5′ Flap probe/FREP probe pair is designed for each target, so multiplex reaction could be detected on different barcoded labeled microbeads in a single sample.

In the first reaction, two oligonucleotides—a probe (consists of 5′ Flap portion of the probe and target specific region) and an Invader oligo (Third Wave Technologies)—hybridize to the target specific DNA region of interest to generate a one-base overlapping structure at the nucleotide being interrogated. If the sequence is present this triplex structure is recognized and cut at a specific site by Cleavase® enzymes, resulting in the release of 5′ Flap oligonucleotide. Multiple probes cycle rapidly on and off the target during the primary reaction. The number of free 5′ flaps is proportional to the amount of target in the sample, allowing for quantitative detection of genes, chromosomes or infectious agents. This 5′ flap then serves as the “Invader” oligo in the secondary reaction which will be occurred on labeled microbeads. These result in specific cleavage of a labeled oligonucleotide, the FRET Probe, which is specific to one 5′ flap probe. Cleavage of this FRET probe results in the generation of a fluorescent signal. The signal is detected at each specific interval (e.g. each 30 seconds).

### Example 5

### Encoded Microbeads—Based Multiplex Targets Amplification Using Isothermal HDA Assay

Helicase dependent amplification (HDA) (Biohelix Corp) is a method for isothermal amplification of nucleic acids. Like PCR, the HDA reaction selectively amplifies a target sequence defined by two primers. However, unlike PCR, HDA uses an enzyme called a helicase to separate DNA, rather than heat. This allows DNA amplification without the need for themocycling. Each primer pair (e.g. #N) is coupled to specific barcoded labeled microbeads (#N). Genomic DNA samples will be incubated with HDA master mixture and primer pair coupled labeled microbeads. As the ds DNA accumulated on the labeled microbeads, fluorescence increases. For signal detection, EvaGreen (Biotium)) could be used for detection of double stranded amplicons. An increase in DNA product during HDA therefore leads to an increase in fluorescence intensity and is measured at the end of 65° C./115″ extension step of each cycle, thus allowing DNA concentrations to be quantified on a bead with a specific barcode. The end-PCR melt curve analysis could also be done on labeled microbeads.

Protocol for on labeled microbeads tHDA (Thermostable HDA) or RT-HDA Target Amplification, and Signal Detection by Streptavidin-R-PE are described in the following section.

Materials for Target Amplification Process:
1. 1×PBST (Tween-20, 0.1%)
2. nuclease-free Water
3. Forward and Reverse Primer Pair to be conjugated on labeled microbeads: 100 μM/μl, 5′ aminoC6 modified-MWG-Biotech AG.
4. BioHelix IsoAmp® II Universal tHDA Kit (tHDA: thermophilic Helicase-Dependent Amplification) Catalog #H0110S

### Experiment Procedure:

On labeled microbeads PCR/RT-PCR Reaction:
1. Prepare a reaction mix A according to table below. The reaction mix A contains 1× annealing buffer; soluble forward and reverse primers pair for each targets/control; and labeled microbeads mix for each target/control. For Mix A preparation, take enough labeled microbeads into 1.5 ml tube. Remove the PBST buffer, and then add nuclease free H₂O and all component to labeled microbeads except DNA/RNA template. Mix well and aliquot 23 μl volume into PCR tubes.

| Mix A | |
|---|---|
| Forward primer coupled labeled microbeads (50 beads per | 20.5 μl |
| type) in H₂O | |
| 10X Annealing buffer II | 2.5 μl |
| DNA template | — |
| Total volume of Mix A | 23 μl |

2. Add 2 μl template DNA/RNA per PCR tube
3. Prepare Mix B according to table below. For RNA samples, Thermoscript RT will be added for reverse transcription. Aliquot 25 μl Mix B into 96-well flat bottomed plate.

| Mix B for: | EvaGreen tHDA | EvaGreen RT-HDA |
|---|---|---|
| H2O | 8.0 μl | 7.75 μl |
| 10X Annealing buffer II | 2.5 μl | 2.5 μl |
| MgSO4 (100 mM)* | 2 μl | 1.75 μl |
| NaCl (500 mM)* | 4 μl | 4 μl |
| IsoAmp ® dNTP Solution | 3.5 μl | 3.5 μl |
| IsoAmp ® Enzyme Mix | 3.5 μl | 3.5 μl |
| EvaGreen (20X, Biotium) | 0.5 μl | 0.5 μl |
| ROX Reference Dye (50X, | 1 μl | 1 μl |
| Invitrogen) | | |
| ThermoScript RT | 0 | 0.5 μl |
| (2.1 U/μl, Invitrogen)* | | |
| Total volume of Mix B | 25 μl | 25 μl |

4. Place the PCR tubes containing Mix A in the thermal cycler and incubate at 95° C. for 2 minutes and place promptly on ice.
5. Transfer 25 μl of Mix A into Mix B in 96-well plate and gently mix the reaction by pipetting.
6. 60 cycles of 65° C. for 115 seconds, and 66° C. for 5″.
7. Dissociation step: Melt curve data collection and analysis

* The condition of tHDA reactions can be further optimized by titering the
following components:

| following | Components Recommended | | Recommended | |
|---|---|---|---|---|
| components: | concentration | | concentration for titering | |
| MgSO4 | 3.5 to 4 | mM | 3 to 4.5 | mM |
| NaCl | 30 to 40 | mM | 20 to 50 | mM |
| Primer | 75 to 100 | nM | 50 to 200 | nM |
| ThermoScript RT | 1 to 2 | units | 0.5 to 10.5 | units |

### Example 6

### Labeled Microbeads—Based Real-Time Multiplex Targets Amplification Using Multicode—RTx.

MultiCode-RTx is a probe-free, real-time PCR method developed by EraGen which can be adapted for use on labeled microbeads for real-time multiplex PCR application. The forward primer is designed to include a fluorescent reporter-labeled primer with an isoC on the 5′ end and 5′ AminoC12 modified. The reporter labeled primer N will be coupled on labeled microbeads N. The synthesis of double strand amplicon will incorporate an isoG with a covalently attached quencher molecule. The resulting proximity of the quencher to the reporter produces a decrease in fluorescence on labeled microbeads. The fluorescence decrease is directly proportional to the amount of amplicon. The end-PCR melt curve analysis could also be done on labeled microbeads. The fluorescence is restored after the double strand separate.

### Labeled Microbeads—Based Multiplex PCR Analyzer

The labeled microbeads—based multiplex PCR analyzer is similar to the conventional real-time PCR system, but includes a sample plate which has a flat bottom for steady state optical bead imaging. The sample plates, such as clear flat bottom 8-well strip, 96-well or 384-well microplate, are suited for light illumination and image detection. After each cycle, both barcode image and fluorescence image are taken when the beads are at the bottom of the plate. It takes about 5-10 seconds for the beads to settle down to the bottom of the plate. The increasing amount of emitted fluorescence is proportional to the increasing amount of DNA generated during the linear phase of the ongoing PCR process. FIG. 5 shows the images of the labeled microbeads at the bottom of the flat bottom microplate. In a typical assay, fluorescent intensity measurements are made after the annealing steps. After each cycle of annealing step or extension step, or after selected periods of time beads are decoded by image processing and detected by fluorescence. Tens, hundreds, or thousands of beads can be monitored simultaneously in a single microwell.

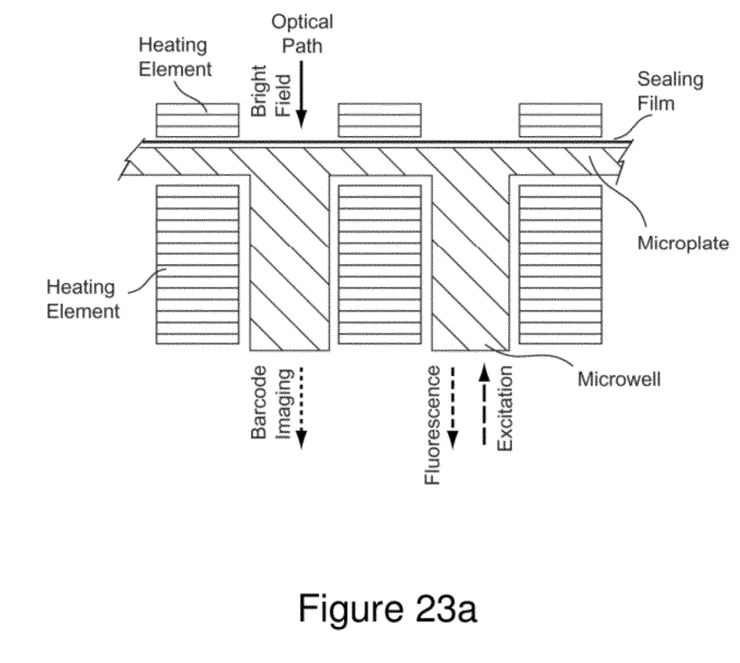

FIG. 22 shows the typical PCR steps with labeled microbeads tagged probes. PCR involves the following three steps: denaturation, annealing and extension. First, the genetic material is denatured, converting the double stranded DNA molecules to single strands. The primers are then annealed to the complementary regions of the single stranded molecules. In the third step, they are extended by the action of the DNA polymerase. All these steps are temperature sensitive and the common choice of temperatures is 94° C., 60° C. and 70° C. respectively. Optical detection is performed after the annealing or extension step, the labeled microbeads will be settled down to the bottom of the plate for optical imaging. Modern thermal cyclers are equipped with a heated lid, a heated plate that presses against the lids of the reaction tubes. This prevents condensation of water from the reaction mixtures on the insides of the lids and makes it unnecessary to use PCR oil to cover the reaction mixture. Some thermal cyclers are equipped with multiple blocks allowing several different PCR reactions to be carried out simultaneously.

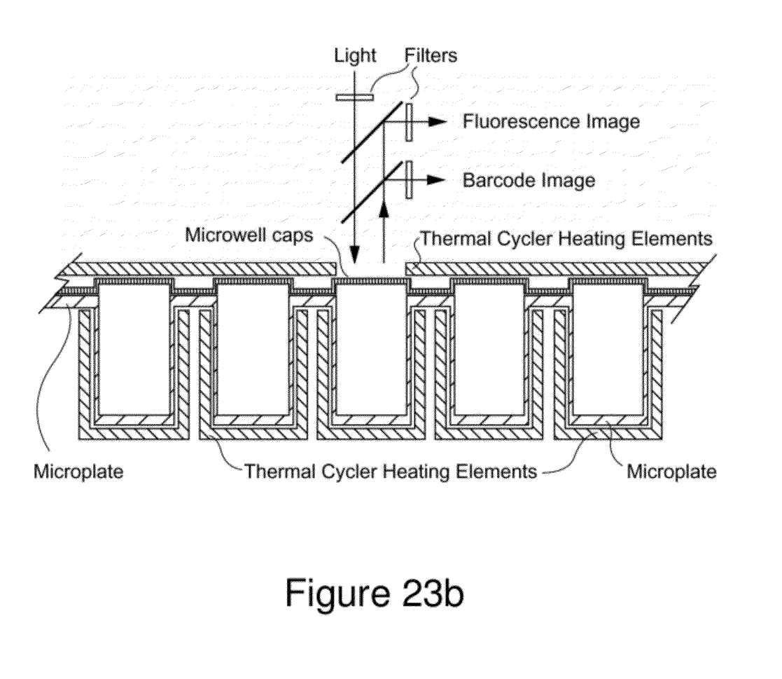

FIG. 23a shows the heating and cooling elements of the labeled microbeads-based multiplex real-time PCR system. The heating element or thermocycling block encloses the microwell and has open area for light illumination and optical detection. The opening area can have mechanical mechanism, therefore they are normally closed. Quality thermal cyclers often contain silver blocks to achieve fast temperature changes and uniform temperature throughout the block. Also some apparatus have a gradient function, which allows different temperatures in different parts of the block. This is particularly useful when testing suitable annealing temperatures for primers. A typical thermocycle requires only 30 to 60 seconds; an amplification reaction with 30 cycles is usually complete in about 30 minutes. To avoid contamination, the capillary is tightly closed such that it need not be opened at any time during analysis. Heating and cooling can be achieved through various rapid heating/cooling sources. Peltier pump and airflow are the commonly used in the PCR system to control the heating cycle. Sample plates or tubes are inserted into the heating block controlled by the Peltier pump. Hot and cold airflow are also used to heat or cool the sample chamber.

To enhance the surface chemistry, microbeads can be mixed in solution by various methods, such as rotation, shaking, acoustic wave, magnetic field, and ultrasound mixing. Electromagnetic devices are used for magnetic beads mixing in the automatic robotic systems. Acoustic wave and ultrasound are often used for liquid mixing. Sample plate or sample microwell should be sealed with an optically clear sealing film before put into themocycler.

Alternatively, FIG. 23b shows the optical detection can be performed from the top of the microplate. Both barcode image and fluorescence image can be obtained based on reflection mode. Two optical filters are used to separate the reflective barcode image and fluorescence image. The optical detection system can rapidly scan the microwell during or after PCR cycles.

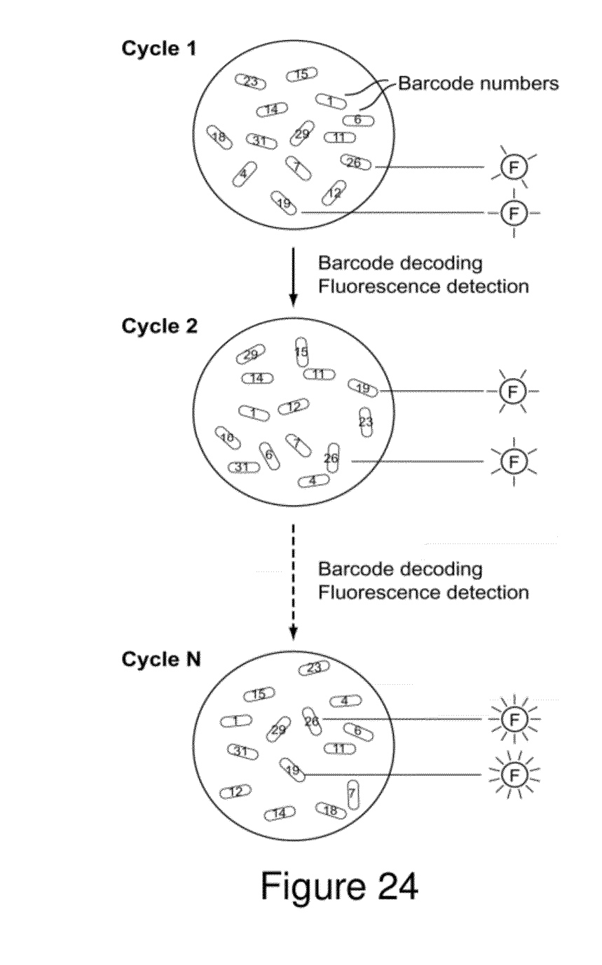

Every single bead is decoded and fluorescence detected. Each type of probe or primer can be tagged on a few beads. FIG. 24 shows the fluorescence signal increase as function of the amplification cycle and detected on the bottom of the microplate. Due to the gravity effect, beads are settled down to the bottom of the plate in 3-10 seconds. The optical paths of both bright field and fluorescence are collinear. The fluorescence signal is monitored with respected to each labeled microbeads with a specific barcode in a microwell. Every single bead is decoded and fluorescently detected. The microplate holder has an XY translational stage, which rapidly scans the entire microplate. Depend on the optical configuration, either top or bottom (or both) of the well is optically accessible.

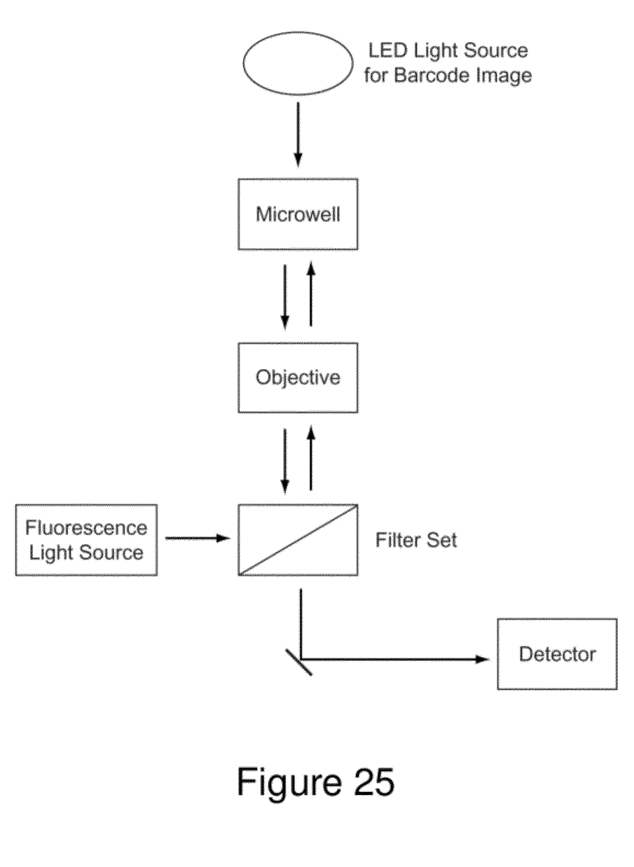

FIG. 25 shows the optical components of the real-time analytical system. A 0.5 mw LED is sufficient for bright field top illumination in transmission or reflection mode for barcode imaging and identification. High power LED, mercury lamp, metal halide lamp or lasers are common light source for bottom fluorescence excitation in reflection mode. The resolution of the bar code image (5 μm) is optimized optically with a 4×−10× objective lens, so that decoding information can be detected with sufficient spatial resolution. A CCD is used alternately for both bright field barcode imaging and fluorescence detection (reflection mode).

Because of their thin width, beads lie flat on the bottom of the well. (Orientation does not matter). The CCD scans the whole microwell. The 4× objective has a field of view 6.5 mm diameter, while 10× objective has 1 mm diameter. The amount of time for both barcode imaging and fluorescence image is approximately 1 second per frame. By measuring the integrated fluorescence intensity from each bead, one can monitor the experimental results based on the biochemical kinetics. Depend of the fluorescence dye used, a selected filter set removes the illumination light source and pass through the reflective fluorescence light. By decoding the digital bar codes, one classify which biological probe is immobilized on the bead surface. The machine contains a sensitive camera that monitors the fluorescence in each well of the 96-well plate at frequent intervals during the PCR Reaction.

A large number of barcoded beads can be measured simultaneously. The current labeled microbeads have 7-digit and 10-digit barcodes, which represent 128 and 1,024 barcodes, respectively. Therefore, it is possible to perform 128-plex or 1,024-plex real-time PCR reactions in a sample. The barcode 0000000000 represents the barcode number 0, while barcode 1111111111 represents the barcode number 1,023. The labeled microbeads is made of transparent polymer, both barcode and fluorescence can be detected from both sides of the surfaces. The bead has three layers wherein the magnetic material is sandwiched between the two polymer layers. PCR reaction occurs on the surface of the microbeads. The fluorescence data for each bead is output after each amplification cycle.

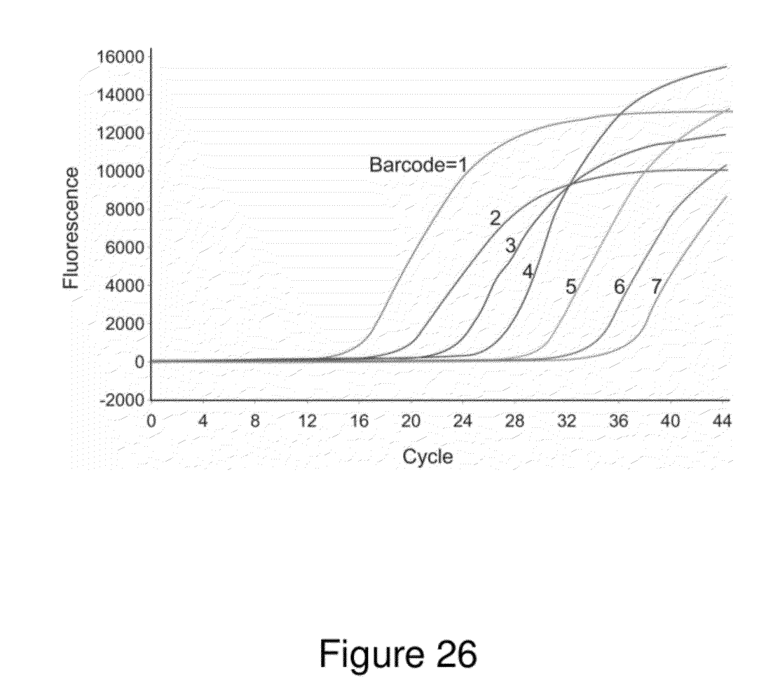

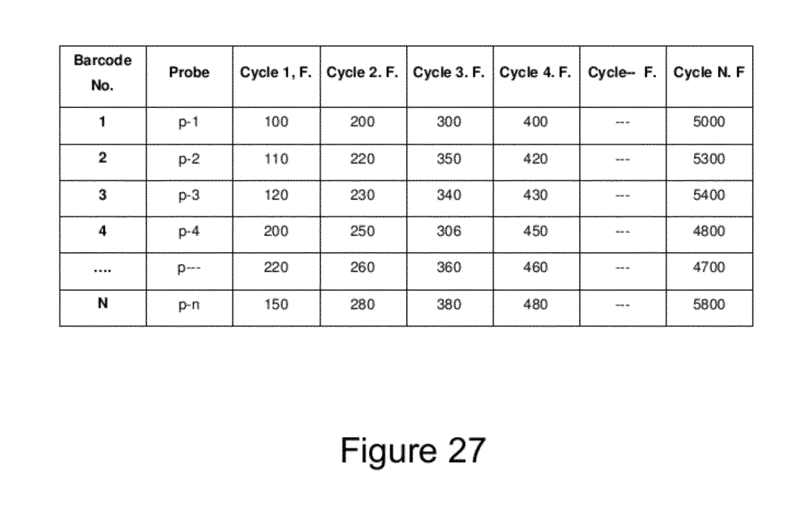

FIGS. 26 and 27 show the fluorescence signal and data as function of the number of cycle for different labeled microbeads tagged with different probes. Depending on the target template and its quantity, the fluorescence signals can be monitored based on every single bead. The recognition of the specific probe or primer can be determined by the barcode. The amount of DNA theoretically doubles with every cycle of PCR. After each cycle, the amount of DNA is twice what it was before. Thus, after N cycles one shall have 2N times as much. But, of course, the reaction cannot go on forever, and it eventually tails off and reaches a plateau phase. If one plots these figures in the standard fashion, one cannot detect the amplification in the earlier cycles because the changes do not show up on this scale. Eventually one sees the last few cycles of the linear phase as they rise above the baseline and then the non-linear or plateau phase. If one plots these values on a logarithmic scale, one can see the small differences at earlier cycles. In real time PCR one uses both types of graph to examine the data. Note that there is a straight line relationship between the amount of DNA and cycle number when one looks on a logarithmic scale. This is because PCR amplification is an exponential reaction.

In summary, by combining encoded microbeads and real-time PCR amplification, it is possible to increase the multiplexity of PCR experiment to a very large number. No more limitation based on the number of fluorophore available. Oligonucleotide probe or primer tagged with encoded microbeads offers the ability to screen and identify hundreds or thousands of various biological targets, different alleles and microbial organisms simultaneously.

Numerous modifications and variations in the practice of the invention are expected to occur to those skilled in the art upon consideration of the presently preferred embodiments thereof. Consequently, the only limitations which should be placed upon the scope of the invention are those which appear in the appended claims.

What is claimed is:

1. An amplification method for the analysis of nucleic acid samples comprising the steps of:
contacting said sample with a nucleic acid primer or probe capable of hybridizing with a selected target nucleic acid under chain extension conditions wherein said primer or probe is linked to a microbead presenting an optically detectable code specific for said primer or probe;
conducting polymerase mediated chain extension such that the presence of target nucleic acid results in the presence or absence of a detectable signal physically associated with the encoded microbead;
settling said encoded microbeads to the bottom of a well; and
measuring the signal associated with said encoded microbead at more than one point in time.

2. The method of claim 1, wherein the encoded beads are encoded with a barcode.

3. The method of claim 2 wherein the encoded beads have 2 to 4,096 different barcode patterns.

4. The method of claim 1, wherein the encoded beads are chemically and physically stable up to 98° C.

5. The method of claim 1, wherein the encoded beads are smaller than 1 mm in length.

6. The method of claim 1, wherein the encoded beads are illuminated with a light source having a wavelength between 400-750 nm to obtain the barcode image and fluorescence image of said encoded beads.

7. The method of claim 2 wherein the barcoded beads have from 2 to 4,096 different barcode patterns.

8. The method of claim 1 wherein the beads are magnetic beads.

9. The method of claim 1, wherein the encoded microbeads comprise: a body comprising an intermediate layer sandwiched between two layers of a photoresist photopolymer material wherein the intermediate layer is coated or imbedded with a paramagnetic material and comprises an encoded pattern which is partially substantially transmissive and partially substantially opaque to light, wherein said pattern provides a code corresponding to the micro bead, wherein the outermost surface of the micro bead comprises said photoresist photopolymer and said photoresist photopolymer is functionalized with a target or captures a molecule selected from the group consisting of proteins, nucleic acids and small molecules.

10. The method of claim 9, wherein the intermediate layer comprises a series of alternating substantially light transmissive sections and substantially light opaque sections defining the encoded pattern, wherein the light transmissive sections are defined by slits through the intermediate layer of the body, and the light opaque sections are defined by a light reflective material or a light absorptive material; wherein the slits comprises slits of a first width and slits of a second width, and wherein the first width represents a “0” and the second width representing a “1” in a binary code.

11. The method of claim 8 wherein a magnetic field is used to settle said encoded microbeads to the bottom of a well.

12. The method of claim 1 further comprising multiple cycles of thermally denaturing and annealing double stranded nucleic acids.

13. The method of claim 12 wherein the signal associated with said encoded microbeads is measured at the same point of each two or more thermal cycles.

14. The method of claim 13 wherein said sample is contacted with helicase and said target nucleic acid is amplified by helicase dependent amplification.

15. The method of claim 14 wherein the signal associated with said encoded microbeads is measured at the multiple selected points in time.

16. A system for carrying out real-time polymerase chain reaction (PCR) detecting for analyzing multiple target molecules in a thermal cycler, the system comprising:
a. a plurality of samples, each sample containing multiple encoded microbeads, a fluorescence primer adapted to target molecules, each encoded microbead having a specific barcode pattern;
b. each sample in a respective one of a plurality of sample wells, each sample well having a flat surface;
c. a thermal cycler;
d. one or two light beams to illuminate and take a barcode optical image and a fluorescence image when said encoded microbeads settle down to the flat surface of said sample wells; and
e. an image software to decode the specific barcode pattern of each encoded microbead and measure the fluorescence intensity of each encoded microbead during a PCR reaction.
